(19) United States
(12) Patent Application Publication
(10) Pub. No.: US 2021/0188813 A1
(43) Pub. Date: Jun. 24, 2021

(54) EZH2 INHIBITOR AND PHARMACEUTICALLY ACCEPTABLE SALTS AND POLYMORPHIC SUBSTANCES THEREOF, AND APPLICATION OF EZH2 INHIBITOR

(71) Applicants: SHANGHAI HAIYAN PHARMACEUTICAL TECHNOLOGY CO., LTD., Shanghai (CN); YANGTZE RIVER PHARMACEUTICAL GROUP CO., LTD., Jiangsu (CN)

(72) Inventors: Taotao JIANG, Shanghai (CN); Shuangni ZHAO, Shanghai (CN); Jinjing LI, Shanghai (CN); Xia YAO, Shanghai (CN)

(21) Appl. No.: 17/050,412

(22) PCT Filed: Apr. 23, 2019
(86) PCT No.: PCT/CN2019/083971

§ 371 Date: Oct. 23, 2020

(30) Foreign Application Priority Data
Apr. 24, 2018 (CN) 201810372377.8

(51) Int. Cl. C07D 401/14

(52) U.S. Cl. CPC C07D 401/14; C07B 2200/13

(57) ABSTRACT

The present invention provides an EZH2 inhibitor and pharmaceutically acceptable salts and polymorphic substances thereof, and application of the EZH2 inhibitor. Specifically, the present invention provides N-((4,6-dimethyl-2-oxo-1,2-dihydropyridin-3-yl)methyl)-5-ethyl-4-(ethyl((1S,4S)-4-(3-methoxyazetidin-1-yl)cyclohexyl)amino)-1-methyl-1H-indazole-6-carboxamide or polymorphic substances of pharmaceutically acceptable salts thereof, and application of N-((4,6-dimethyl-2-oxo-1,2-dihydropyridin-3-yl)methyl)-5-ethyl-4-(ethyl((1S,4S)-4-(3-methoxyazetidin-1-yl)cyclohexyl)amino)-1-methyl-1H-indazole-6-carboxamide. In addition, also disclosed are a pharmaceutical composition containing the inhibitor and application thereof.

### TECHNICAL FIELD

The present invention belongs to the technical field of medicine. In particular, the present invention relates to a EZH2 inhibitor and pharmaceutically acceptable salts and polymorphs thereof and their applications, and the inhibitor is N-((4,6-dimethyl-2-oxo-1,2-dihydropyridin-3-yl)methyl)-5-ethyl-4-(ethyl((1S,4S)-4-(3-methoxyazetidin-1-yl)cyclohexyl)amino)-1-methyl-1H-indazole-6-carboxamide.

### BACKGROUND

The histone-lysine-N-methyltransferase EZH2 is involved in DNA methylation and final transcription inhibition; the methylation of lysine at position 27 is catalyzed by the cofactor S-adenosyl-L-methionine to histidine H3. This methylation promotes the formation of heterochromatin, which triggers gene silencing. EZH2 is part of the PRC2 functional enzyme, and a gene that maintains, controls and regulates development and differentiation through epigenetics, thus ensuring the healthy development of the embryo. The mutation or overexpression of EZH2 is associated with the formation of many cancers. The EZH2 controlling gene controls the development of the tumor, the inhibition of EZH2 activity will slow the growth of the tumor. As a target inhibitor, EZH2 can regulate a variety of cancers including breast cancer, prostate cancer, melanoma and bladder cancer. The PCT applications WO2011140324A1 and WO2012075080A1 disclose indole derivatives as EZH2 inhibitors for treating cancer. The PCT application WO2012118812A2 discloses bicyclic heterocyclic compounds as EZH2 inhibitors for the treatment of cancer.

Therefore, the inhibition of EZH2 activity will effectively reduce cell proliferation and invasion, thereby providing a beneficial treatment for EZH2-mediated diseases or conditions. The compounds of the invention provide solutions for treatment of diseases or EZH2-mediated tumor as EZH2 inhibitors. The present invention develops various salt forms an Form D crystals of the EZH2 inhibitor on the basis of the foregoing work, which is helpful for further drug development.

### SUMMARY OF THE INVENTION

The object of the present invention is to provide pharmaceutically acceptable salts of an EZH2 inhibitor and polymorphs and applications thereof.

In the first aspect of the present invention, a pharmaceutically acceptable salt of a compound of formula X, a polymorph of the compound of formula X, or a polymorph of the pharmaceutically acceptable salt of the compound of formula X is provided:

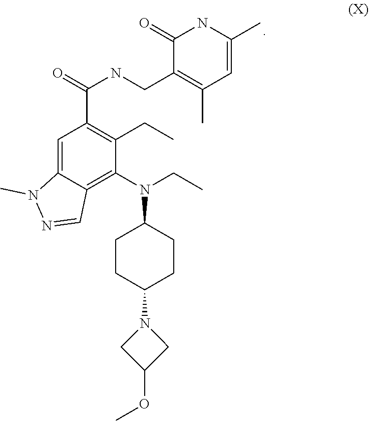

In another preferred example, the pharmaceutically acceptable salt is selected from the group consisting of hydrochloride, sulfate, phosphate, maleate, fumarate, L-tartrate, citrate, methanesulfonate or hydrobromide.

In another preferred example, the pharmaceutically acceptable salt of the compound of formula X or the polymorph of the compound of formula X and its pharmaceutically acceptable salt is in an anhydrous form, hydrate form or solvate form.

In another preferred example, the pharmaceutically acceptable salt is selected from the group consisting of hydrochloride, maleate.

In another preferred example, the pharmaceutically acceptable salt is hydrochloride, and the molar ratio of hydrochloric acid to the compound of formula X is (0.8-2.1):1, preferably (0.9-1.1):1.

In another preferred example, the pharmaceutically acceptable salt is maleate, and the molar ratio of maleic acid to the compound of formula X is (0.8-1.2):1, preferably (0.9-1.1):1, more preferably 1:1.

In another preferred example, the polymorph is Form A crystal of the hydrochloride of compound of formula X, i.e. crystal form A, the X-ray powder diffraction pattern of which has peaks at diffraction angles 2θ(°) values of the following group A1: 4.64±0.20, 9.31±0.20, 12.11±0.20, 12.45±0.20, 13.24±0.20, 14.44±0.20, 15.28±0.20, 16.24±0.20, 16.42±0.20, 22.63±0.20, 37.87±0.20.

In another preferred example, the X-ray powder diffraction pattern of the crystal form A further has peaks at 2 or more than 2 of diffraction angles 2θ (°) values selected from the following group A2: 24.10±0.20, 24.23±0.20, 26.86±0.20, 27.13±0.20, 38.32±0.20, 44.08±0.20.

In another preferred example, the X-ray powder diffraction pattern of the crystal form A further has peaks at 2 or more than 2 of diffraction angles 2θ (°) values selected from the following group A3: 1.06±0.20, 1.30±0.20, 8.84±0.20, 11.24±0.20, 13.68±0.20, 20.80±0.20, 21.86±0.20, 24.58±0.20, 25.12±0.20, 25.39±0.20, 29.42±0.20, 30.65±0.20, 33.23±0.20.

In another preferred example, the X-ray powder diffraction pattern of the crystal form A has peaks at 6 or more or all (such as 6, 7, 8, 9, 10, 11, 12, 13, 14, 15, etc.) of 2θ (°) values selected from the groups A1, A2 and A3.

In another preferred example, the X-ray powder diffraction pattern of the crystal form A has peaks at 2θ(°) values shown in table A1 and the relative intensity of each peak is as shown in table A1:

TABLE A1

| 2θ(°) | I/I₀ | 2θ(°) | I/I₀ | 2θ(°) | I/I₀ |
|---|---|---|---|---|---|
| 1.06 | W | 1.30 | W | 4.64 | S |
| 8.84 | W | 9.31 | VS | 11.24 | W |
| 12.11 | VS | 12.45 | S | 13.24 | S |
| 13.68 | W | 14.44 | S | 15.28 | S |
| 16.24 | S | 16.42 | S | 20.80 | W |
| 21.86 | W | 22.63 | S | 24.10 | M |
| 24.23 | M | 24.58 | W | 25.12 | W |
| 25.39 | W | 26.86 | M | 27.13 | M |
| 29.42 | W | 30.65 | W | 33.23 | W |
| 37.87 | S | 38.32 | M | 44.08 | M |

In another preferred example, the X-ray powder diffraction pattern of crystal form A is substantially as shown in FIG. 1.

In another preferred example, in the crystal form A, the molar ratio of hydrochloric acid to the compound of formula X is (0.8-2.1):1, preferably (0.9-1.1):1, more preferably 0.9:1.

In another preferred example, the peak temperature of exothermic peak of crystal form A is 198.15° C. (see FIG. 2). TGA results show that the weight loss is 5.471% before 100° C. and the weight loss is 27.259% from 100-295.2° C. (see FIG. 3).

In another preferred example, the polymorph is Form B crystal of the maleate of compound of formula X, i.e. crystal form B, the X-ray powder diffraction pattern of which has peaks at diffraction angles 2θ(°) values of the following group B1:5.95±0.20, 13.96±0.20, 16.09±0.20, 16.42±0.20, 17.77±0.20, 19.03±0.20, 20.29±0.20, 20.65±0.20, 21.73±0.20.

In another preferred example, the X-ray powder diffraction pattern of the crystal form B further has peaks at 2 or more than 2 of diffraction angles 2θ (°) values selected from the following group B12: 10.30±0.20, 11.98±0.20, 22.21±0.20, 23.38±0.20, 24.04±0.20, 27.43±0.20, 37.93±0.20.

In another preferred example, the X-ray powder diffraction pattern of the crystal form B further has peaks at 2 or more than 2 of diffraction angles 2θ (°) values selected from the following group B13: 1.07±0.20, 8.86±0.20, 9.43±0.20, 11.02±0.20, 12.48±0.20, 14.91±0.20, 15.25±0.20, 18.47±0.20, 21.03±0.20, 25.14±0.20, 25.72±0.20, 26.81±0.20, 28.30±0.20, 28.53±0.20, 29.06±0.20, 29.38±0.20, 30.22±0.20, 34.45±0.20, 34.78±0.20, 36.41±0.20, 38.40±0.20, 39.66±0.20, 40.20±0.20, 44.07±0.20, 45.79±0.20.

In another preferred example, the X-ray powder diffraction pattern of the crystal form B has peaks at 6 or more or all (such as 6, 7, 8, 9, 10, 11, 12, 13, 14, 15, etc.) of 2θ(°) values selected from the groups B1, B2 and B3.

In another preferred example, the X-ray powder diffraction pattern of the crystal form B has peaks at 2θ(°) values shown in table B1, and the relative intensity of each peak is as shown in table B1:

TABLE B1

| 2θ(°) | I/I₀ | 2θ(°) | I/I₀ | 2θ(°) | I/I₀ |
|---|---|---|---|---|---|
| 1.07 | W | 5.95 | S | 8.86 | W |
| 9.43 | W | 10.30 | M | 11.02 | W |
| 11.98 | M | 12.48 | W | 13.96 | VS |
| 14.91 | W | 15.25 | W | 16.09 | S |
| 16.42 | VS | 17.77 | S | 18.47 | W |
| 19.03 | S | 20.29 | S | 20.65 | S |
| 21.03 | W | 21.73 | S | 22.21 | M |
| 23.38 | M | 24.04 | M | 25.14 | W |
| 25.72 | W | 26.81 | W | 27.43 | M |
| 28.30 | W | 28.53 | W | 29.06 | W |
| 29.38 | W | 30.22 | W | 34.45 | W |
| 34.78 | W | 36.41 | W | 37.93 | M |
| 38.40 | W | 39.66 | W | 40.20 | W |
| 44.07 | W | 45.79 | W | | |

In another preferred example, the X-ray powder diffraction pattern of crystal form B is substantially as shown in FIG. 4.

In another preferred example, in the crystal form B, the molar ratio of maleic acid to the compound of formula X is (0.8-1.2):1, preferably (0.9-1.1):1, more preferably 1:1.

In another preferred example, the crystal form B has no obvious endothermic and exothermic peaks (see FIG. 5). TGA results showed that the crystal form lost 2.941% before 100° C., and lost 1.214% from 111.97-165.04° C., and lost 6.395% from 165.04-295.43° C. (see FIG. 6).

In another preferred example, the polymorph is Form C crystal of the sulfate of compound of formula X, i.e. crystal form C, the X-ray powder diffraction pattern of which has peaks at diffraction angles 2θ(°) values of the following group C1: 7.00±0.20, 13.18±0.20, 14.14±0.20, 14.44±0.20, 14.62±0.20, 17.65±0.20, 17.81±0.20, 18.11±0.20, 20.44±0.20, 21.85±0.20, 23.89±0.20.

In another preferred example, the X-ray powder diffraction pattern of the crystal form C further has peaks at 2 or more than 2 of diffraction angles 2θ (°) values selected from the following group C2: 22.15±0.20, 25.40±0.20, 27.23±0.20.

In another preferred example, the X-ray powder diffraction pattern of the crystal form C further has peaks at 2 or more than 2 of diffraction angles 2θ (°) values selected from the following group C3: 1.12±0.20, 1.54±0.20, 1.87±0.20, 37.96±0.20.

In another preferred example, the X-ray powder diffraction pattern of the crystal form C has peaks at 6 or more or all (such as 6, 7, 8, 9, 10, 11, 12, 13, 14, 15, etc.) of 2θ (°) values selected from the groups C1, C2 and C3.

In another preferred example, the X-ray powder diffraction pattern of the crystal form C has peaks at 2θ(°) values shown in table C1, and the relative intensity of each peak is as shown in table C1:

TABLE C1

| 2θ(°) | I/I₀ | 2θ(°) | I/I₀ | 2θ(°) | I/I₀ |
|---|---|---|---|---|---|
| 1.12 | W | 1.54 | W | 1.87 | W |
| 7.00 | S | 13.18 | VS | 14.14 | S |
| 14.44 | VS | 14.62 | S | 17.65 | S |
| 17.81 | S | 18.11 | S | 20.44 | S |
| 21.85 | S | 22.15 | M | 23.89 | S |
| 25.40 | M | 27.23 | M | 37.96 | W |

In another preferred example, the X-ray powder diffraction pattern of crystal form C is substantially as shown in FIG. 7.

In another preferred example, the polymorph is Form D crystal of the hydrobromide of compound of formula X, i.e. crystal form D, the X-ray powder diffraction pattern of which has peaks at diffraction angles 2θ(°) values of the following group D1: 14.65±0.20, 16.47±0.20, 17.62±0.20, 17.92±0.20, 21.91±0.20, 22.99±0.20, 23.12±0.20, 24.88±0.20.

In another preferred example, the X-ray powder diffraction pattern of the crystal form D further has peaks at 2 or more than 2 of diffraction angles 2θ (°) values selected from the following group D2: 14.08±0.20, 16.18±0.20, 19.16±0.20, 19.34±0.20, 19.51±0.20, 21.53±0.20, 26.41±0.20.

In another preferred example, the X-ray powder diffraction pattern of the crystal form D further has peaks at 2 or more than 2 of diffraction angles 2θ (°) values selected from the following group D3: 1.04±0.20, 1.24±0.20, 7.96±0.20, 8.80±0.20, 19.73±0.20, 22.09±0.20, 23.35±0.20, 25.92±0.20, 26.24±0.20, 28.16±0.20, 28.46±0.20, 28.63±0.20, 30.48±0.20, 30.62±0.20.

In another preferred example, the X-ray powder diffraction pattern of the crystal form D has peaks at 6 or more or all (such as 6, 7, 8, 9, 10, 11, 12, 13, 14, 15, etc.) of 2θ (°) values selected from the groups D1, D2 and D3.

In another preferred example, the X-ray powder diffraction pattern of the crystal form D has peaks at 2θ(°) values shown in table D1, and the relative intensity of each peak is as shown in table D1:

TABLE D1

| 2θ(°) | I/I₀ | 2θ(°) | I/I₀ | 2θ(°) | I/I₀ |
|---|---|---|---|---|---|
| 1.04 | W | 1.24 | W | 7.96 | W |
| 8.80 | W | 14.08 | M | 14.65 | VS |
| 16.18 | M | 16.47 | S | 17.62 | S |
| 17.92 | S | 19.16 | M | 19.34 | M |
| 19.51 | M | 19.73 | W | 21.53 | M |
| 21.91 | S | 22.09 | W | 22.99 | S |
| 23.12 | S | 23.35 | W | 24.88 | VS |
| 25.92 | W | 26.24 | W | 26.41 | M |
| 28.16 | W | 28.46 | W | 28.63 | W |
| 30.48 | W | 30.62 | W | | |

In another preferred example, the X-ray powder diffraction pattern of crystal form D is substantially as shown in FIG. 8.

In another preferred example, the polymorph is Form E crystal of the methanesulfonate of compound of formula X, i.e. crystal form E, the X-ray powder diffraction pattern of which has peaks at diffraction angles 2θ(°) values of the following group E1: 9.48±0.20, 10.30±0.20, 12.03±0.20, 12.79±0.20, 13.90±0.20, 16.09±0.20, 16.39±0.20, 17.76±0.20, 18.97±0.20, 19.11±0.20, 20.08±0.20, 20.39±0.20, 20.59±0.20, 21.73±0.20, 21.91±0.20, 22.14±0.20, 22.99±0.20, 23.14±0.20, 23.57±0.20, 25.67±0.20.

In another preferred example, the X-ray powder diffraction pattern of the crystal form E further has peaks at 2 or more than 2 of diffraction angles 2θ (°) values selected from the following group E2: 5.48±0.20, 5.81±0.20, 8.78±0.20, 14.54±0.20, 24.69±0.20, 27.28±0.20, 38.03±0.20.

In another preferred example, the X-ray powder diffraction pattern of the crystal form E further has peaks at 2 or more than 2 of diffraction angles 2θ (°) values selected from the following group E3: 1.15±0.20, 1.27±0.20, 1.75±0.20, 36.53±0.20.

In another preferred example, the X-ray powder diffraction pattern of the crystal form E has peaks at 6 or more or all (such as 6, 7, 8, 9, 10, 11, 12, 13, 14, 15, etc.) of 2θ (°) values selected from the groups E1, E2 and E3.

In another preferred example, the X-ray powder diffraction pattern of the crystal form E has peaks at 2θ(°) values shown in table E1, and the relative intensity of each peak is as shown in table E1:

TABLE E1

| 2θ(°) | I/I₀ | 2θ(°) | I/I₀ | 2θ(°) | I/I₀ |
|---|---|---|---|---|---|
| 1.15 | W | 1.27 | W | 1.75 | W |
| 5.48 | M | 5.81 | M | 8.78 | M |
| 9.48 | S | 10.30 | S | 12.03 | S |
| 12.79 | S | 13.90 | VS | 14.54 | M |
| 16.09 | VS | 16.39 | S | 17.76 | VS |
| 18.97 | S | 19.11 | S | 20.08 | S |
| 20.39 | VS | 20.59 | S | 21.73 | S |
| 21.91 | VS | 22.14 | S | 22.99 | VS |
| 23.14 | VS | 23.57 | S | 24.69 | M |
| 25.67 | S | 27.28 | M | 36.53 | W |
| 38.03 | M | | | | |

In another preferred example, the X-ray powder diffraction pattern of crystal form E is substantially as shown in FIG. 9.

In another preferred example, the polymorph is Form F crystal of the L-tartrate of compound of formula X, i.e. crystal form F, the X-ray powder diffraction pattern of which has peaks at diffraction angles 2θ(°) values of the following group F: 10.92±0.20, 11.11±0.20, 11.26±0.20, 15.31±0.20, 16.96±0.20, 17.11±0.20, 18.16±0.20, 18.46±0.20, 20.45±0.20, 23.55±0.20, 25.30±0.20.

In another preferred example, the X-ray powder diffraction pattern of the crystal form F further has peaks at 2 or more than 2 of diffraction angles 2θ (°) values selected from the following group F2: 9.46±0.20, 12.02±0.20, 16.70±0.20, 19.71±0.20, 23.34±0.20, 25.77±0.20, 37.94±0.20.

In another preferred example, the X-ray powder diffraction pattern of the crystal form F further has peaks at 2 or more than 2 of diffraction angles 2θ (°) values selected from the following group F3: 1.04±0.20, 1.84±0.20, 6.19±0.20.

In another preferred example, the X-ray powder diffraction pattern of the crystal form F has peaks at 6 or more or all (such as 6, 7, 8, 9, 10, 11, 12, 13, 14, 15, etc.) of 2θ (°) values selected from the groups F1, F2 and F3.

In another preferred example, the X-ray powder diffraction pattern of the crystal form F has peaks at 2θ(°) values shown in table F1, and the relative intensity of each peak is as shown in table F1:

TABLE F1

| 2θ(°) | I/I₀ | 2θ(°) | I/I₀ | 2θ(°) | I/I₀ |
|---|---|---|---|---|---|
| 1.04 | W | 1.84 | W | 6.19 | W |
| 9.46 | M | 10.92 | S | 11.11 | VS |
| 11.26 | VS | 12.02 | M | 15.31 | VS |
| 16.70 | M | 16.96 | S | 17.11 | S |
| 18.16 | S | 18.46 | S | 19.71 | M |
| 20.45 | S | 23.34 | M | 23.55 | S |
| 25.30 | S | 25.77 | M | 37.94 | M |

In another preferred example, the X-ray powder diffraction pattern of crystal form F is substantially as shown in FIG. 10.

In another preferred example, the polymorph is Form G crystal of the citrate of compound of formula X, i.e. crystal form G, the X-ray powder diffraction pattern of which has peaks at diffraction angles 2θ(°) values of the following group G1: 11.11±0.20, 15.22±0.20, 16.99±0.20, 18.16±0.20, 20.47±0.20, 23.26±0.20, 23.44±0.20.

In another preferred example, the X-ray powder diffraction pattern of the crystal form G further has peaks at 2 or more than 2 of diffraction angles 2θ (°) values selected from the following group G2: 6.01±0.20, 7.75±0.20, 9.49±0.20, 9.64±0.20, 12.04±0.20, 14.71±0.20, 19.06±0.20, 19.45±0.20, 19.57±0.20, 20.24±0.20, 23.83±0.20.

In another preferred example, the X-ray powder diffraction pattern of the crystal form G further has peaks at 2 or more than 2 of diffraction angles 2θ (°) values selected from the following group G3: 1.06±0.20, 1.50±0.20, 8.41±0.20, 9.17±0.20, 17.53±0.20, 21.91±0.20, 26.34±0.20, 28.78±0.20, 29.21±0.20, 30.84±0.20, 33.95±0.20, 37.96±0.20, 38.38±0.20.

In another preferred example, the X-ray powder diffraction pattern of the crystal form G has peaks at 6 or more or all (such as 6, 7, 8, 9, 10, 11, 12, 13, 14, 15, etc.) of 2θ(°) values selected from the groups G1, G2 and G3.

In another preferred example, the X-ray powder diffraction pattern of the crystal form G has peaks at 2θ(°) values shown in table G1, and the relative intensity of each peak is as shown in table G1:

TABLE G1

| 2θ(°) | I/I₀ | 2θ(°) | I/I₀ | 2θ(°) | I/I₀ |
|---|---|---|---|---|---|
| 1.06 | W | 1.50 | W | 6.01 | M |
| 7.75 | M | 8.41 | W | 9.17 | W |
| 9.49 | M | 9.64 | M | 11.11 | VS |
| 12.04 | M | 14.71 | M | 15.22 | VS |
| 16.99 | S | 17.53 | W | 18.16 | S |
| 19.06 | M | 19.45 | M | 19.57 | M |
| 20.24 | M | 20.47 | S | 21.91 | W |
| 23.26 | S | 23.44 | S | 23.83 | M |
| 26.34 | W | 28.78 | W | 29.21 | W |
| 30.84 | W | 33.95 | W | 37.96 | W |
| 38.38 | W | | | | |

In another preferred example, the X-ray powder diffraction pattern of crystal form G is substantially as shown in FIG. 11.

In another preferred example, the polymorph is crystal form I of the compound of formula X, the X-ray powder diffraction pattern of which has peaks at diffraction angles 2θ(°) values of the group I-1: 7.09±0.20, 9.58±0.20, 11.17±0.20, 13.40±0.20, 14.02±0.20, 14.65±0.20, 16.51±0.20, 17.59±0.20.

In another preferred example, the X-ray powder diffraction pattern of the crystal form I further has peaks at 2 or more than of diffraction angles 2θ(°) values selected from the following group I-2: 18.49±0.20, 19.27±0.20, 20.80±0.20, 22.00±0.20, 24.64±0.20, 24.88±0.20, 25.57±0.20, 25.71±0.20, 28.09±0.20.

In another preferred example, the X-ray powder diffraction pattern of the crystal form I further has peaks at 2 or more than 2 of diffraction angles 2θ (°) values selected from the following group I-3: 1.09±0.20, 1.72±0.20, 8.14±0.20, 11.52±0.20, 15.28±0.20, 22.42±0.20, 22.96±0.20, 26.53±0.20, 27.55±0.20, 28.24±0.20, 29.19±0.20, 30.44±0.20, 30.70±0.20, 34.51±0.20, 36.62±0.20, 37.96±0.20, 38.31±0.20, 47.84±0.20.

In another preferred example, the X-ray powder diffraction pattern of the crystal form I has peaks at 6 or more or all (such as 6, 7, 8, 9, 10, 11, 12, 13, 14, 15, etc.) of 2θ (°) values selected from the groups I-1, I-2 and I-3.

In another preferred example, the X-ray powder diffraction pattern of the crystal form I has peaks at 2θ(°) values shown in table I1, and the relative intensity of each peak is as shown in table I1:

TABLE I1

| 2θ(°) | I/I₀ | 2θ(°) | I/I₀ | 2θ(°) | I/I₀ |
|---|---|---|---|---|---|
| 1.09 | W | 1.72 | W | 7.09 | VS |
| 8.14 | W | 9.58 | S | 11.17 | S |
| 11.52 | W | 13.40 | VS | 14.02 | S |
| 14.65 | VS | 15.28 | W | 16.51 | S |
| 17.59 | VS | 18.49 | M | 19.27 | M |
| 20.80 | M | 22.00 | M | 22.42 | W |
| 22.96 | W | 24.64 | M | 24.88 | M |
| 25.57 | M | 25.71 | M | 26.53 | W |
| 27.55 | W | 28.09 | M | 28.24 | W |
| 29.19 | W | 30.44 | W | 30.70 | W |
| 34.51 | W | 36.62 | W | 37.96 | W |
| 38.31 | W | 47.84 | W | | |

In another preferred example, the X-ray powder diffraction pattern of crystal form I is substantially as shown in FIG. 12.

In another preferred example, the crystal form I has an exothermic peak at 242.01° C. (see FIG. 13); and has a weight loss of 0.3671% from 100° C. to 228.72° C., and a weight loss of 0.2984% from 228.72° C. to 295.25° C. (see FIG. 14).

In another preferred example, the polymorph is crystal form II of the compound of formula X, the X-ray powder diffraction pattern of which has peaks at diffraction angles 2θ(°) values of the following group II-1: 5.32±0.20, 7.11±0.20, 9.16±0.20, 9.56±0.20, 11.15±0.20, 11.62±0.20, 13.45±0.20, 14.02±0.20, 14.65±0.20, 16.54±0.20, 17.62±0.20, 21.91±0.20, 37.96±0.20, 38.38±0.20, 44.20±0.20.

In another preferred example, the X-ray powder diffraction pattern of the crystal form II further has peaks at 2 or more than of diffraction angles 2θ(°) values selected from the following group II-2: 15.94±0.20, 17.03±0.20, 18.49±0.20, 18.85±0.20, 19.18±0.20, 20.35±0.20, 20.77±0.20, 21.80±0.20, 25.60±0.20, 44.67±0.20.

In another preferred example, the X-ray powder diffraction pattern of the crystal form II further has peaks at 2 or more than 2 of diffraction angles 2θ (°) values selected from the following group II-3: 1.09±0.20, 1.30±0.20, 1.66±0.20, 1.84±0.20, 8.17±0.20, 15.28±0.20, 22.57±0.20, 24.44±0.20, 24.64±0.20, 24.87±0.20, 25.18±0.20, 27.29±0.20, 28.01±0.20, 29.05±0.20, 29.42±0.20.

In another preferred example, the X-ray powder diffraction pattern of the crystal form II has peaks at 6 or more or all (such as 6, 7, 8, 9, 10, 11, 12, 13, 14, 15, etc.) of 2θ (°) values selected from the groups II-1, II-2 and II-3.

In another preferred example, the X-ray powder diffraction pattern of the crystal form II has peaks at 2θ(°) values shown in table II1, and the relative intensity of each peak is as shown in table II1:

TABLE II1

| 2θ(°) | I/I₀ | 2θ(°) | I/I₀ | 2θ(°) | I/I₀ |
|---|---|---|---|---|---|
| 1.09 | W | 1.30 | W | 1.66 | W |
| 1.84 | W | 5.32 | S | 7.11 | VS |
| 8.17 | W | 9.16 | S | 9.56 | S |
| 11.15 | S | 11.62 | S | 13.45 | VS |
| 14.02 | S | 14.65 | VS | 15.28 | W |
| 15.94 | M | 16.54 | S | 17.03 | M |
| 17.62 | VS | 18.49 | M | 18.85 | M |
| 19.18 | M | 20.35 | M | 20.77 | M |
| 21.80 | M | 21.91 | S | 22.57 | W |
| 24.44 | W | 24.64 | W | 24.87 | W |
| 25.18 | W | 25.60 | M | 27.29 | W |
| 28.01 | W | 29.05 | W | 29.42 | W |
| 37.96 | VS | 38.38 | S | 44.20 | S |
| 44.67 | M | | | | |

In another preferred example, the X-ray powder diffraction pattern of crystal form II is substantially as shown in FIG. 16.

In another preferred example, the crystal form II has no obvious endothermic exothermic peak (see FIG. 17).

In another preferred example, the polymorph is crystal form III of the compound of formula X, the X-ray powder diffraction pattern of which has peaks at diffraction angles 2θ(°) values of the following group III-1: 6.97±0.20, 9.01±0.20, 9.21±0.20, 12.46±0.20, 14.86±0.20, 15.28±0.20, 15.46±0.20, 20.92±0.20, 22.90±0.20.

In another preferred example, the X-ray powder diffraction pattern of the crystal form III further has peaks at 2 or more than 2 of diffraction angles 2θ (°) values selected from the following group III-2: 12.17±0.20, 13.89±0.20, 16.21±0.20, 18.10±0.20, 19.30±0.20, 20.14±0.20, 24.56±0.20, 24.76±0.20, 27.49±0.20.

In another preferred example, the X-ray powder diffraction pattern of the crystal form III further has peaks at 2 or more than 2 of diffraction angles 2θ (°) values selected from the following group III-3: 1.30±0.20, 1.51±0.20, 9.66±0.20, 10.15±0.20, 17.10±0.20, 22.00±0.20, 23.31±0.20, 25.18±0.20, 26.03±0.20, 26.74±0.20, 28.03±0.20, 29.53±0.20, 31.30±0.20, 31.46±0.20, 33.79±0.20, 34.01±0.20, 35.98±0.20, 37.96±020, 38.38±0.20, 42.10±0.20, 42.25±0.20, 44.23±0.20, 44.66±0.20.

In another preferred example, the X-ray powder diffraction pattern of the crystal form III has peaks at 6 or more or all (such as 6, 7, 8, 9, 10, 11, 12, 13, 14, 15, etc.) of 2θ (°) values selected from the groups III-1, III-2 and III-3.

In another preferred example, the X-ray powder diffraction pattern of the crystal form III has peaks at 2θ(°) values shown in table III1, and the relative intensity of each peak is as shown in table III1:

TABLE III1

| 2θ(°) | I/I₀ | 2θ(°) | I/I₀ | 2θ(°) | I/I₀ |
|---|---|---|---|---|---|
| 1.30 | W | 1.51 | W | 6.97 | VS |
| 9.01 | S | 9.21 | S | 9.66 | W |
| 10.15 | W | 12.17 | M | 12.46 | VS |
| 13.89 | M | 14.86 | S | 15.28 | S |
| 15.46 | S | 16.21 | M | 17.10 | W |
| 18.10 | M | 19.30 | M | 20.14 | M |
| 20.92 | S | 22.00 | W | 22.90 | S |
| 23.31 | W | 24.56 | M | 24.76 | M |
| 25.18 | W | 26.03 | W | 26.74 | W |
| 27.49 | M | 28.03 | W | 29.53 | W |
| 31.30 | W | 31.46 | W | 33.79 | W |
| 34.01 | W | 35.98 | W | 37.96 | W |
| 38.38 | W | 42.10 | W | 42.25 | W |
| 44.23 | W | 44.66 | W | | |

In another preferred example, the X-ray powder diffraction pattern of crystal form III is substantially as shown in FIG. 19.

In another preferred example, the crystal form III has no obvious endothermic and exothermic peak (see FIG. 20) In another preferred example, the polymorph is crystal form IV of the compound of formula X, the X-ray powder diffraction pattern of which has peaks at diffraction angles 2θ(°) values of the following group IV-1: 5.26±0.20, 6.94±0.20, 8.98±0.20, 9.52±0.20, 11.62±0.20, 12.40±0.20, 13.42±0.20, 14.62±0.20, 16.51±0.20, 17.62±0.20, 18.01±0.20, 19.18±0.20, 21.85±0.20, 27.31±0.20.

In another preferred example, the X-ray powder diffraction pattern of the crystal form IV further has peaks at 2 or more than 2 of diffraction angles 2θ (°) values selected from the following group IV-2: 15.35±0.20, 15.99±0.20, 20.86±0.20, 22.81±0.20, 22.96±0.20, 24.72±0.20, 25.15±0.20, 25.41±0.20, 25.63±0.20, 37.92±0.20.

In another preferred example, the X-ray powder diffraction pattern of the crystal form IV further has peaks at 2 or more than 2 of diffraction angles 2θ (°) values selected from the following group IV-3: 1.16±0.20, 1.57±0.20, 10.07±0.20, 11.11±0.20, 13.93±0.20, 16.90±0.20, 18.49±0.20, 18.82±0.20, 20.11±0.20, 22.50±0.20, 24.43±0.20, 26.44±0.20, 26.65±0.20, 28.00±0.20, 29.30±0.20, 29.66±0.20, 34.45±0.20, 34.66±0.20, 36.83±0.20, 38.35±0.20, 44.17±0.20, 44.71±0.20, 47.94±0.20, 48.21±0.20.

In another preferred example, the X-ray powder diffraction pattern of the crystal form IV has peaks at 4 or more or all (such as 4, 5, 6, 7, 8, 9, 10, 11, 12, 13, etc.) of 2θ (°) values selected from the groups IV-1, IV-2 and IV-3.

In another preferred example, the X-ray powder diffraction pattern of the crystal form IV has peaks at 2θ(°) values shown in table IV1, and the relative intensity of each peak is as shown in table IV1:

TABLE IV1

| 2θ(°) | I/I₀ | 2θ(°) | I/I₀ | 2θ(°) | I/I₀ |
|---|---|---|---|---|---|
| 1.16 | W | 1.57 | W | 5.26 | S |
| 6.94 | VS | 8.98 | VS | 9.52 | S |
| 10.07 | W | 11.11 | W | 11.62 | S |
| 12.40 | S | 13.42 | S | 13.93 | W |
| 14.62 | VS | 15.35 | M | 15.99 | M |
| 16.51 | S | 16.90 | W | 17.62 | S |
| 18.01 | S | 18.49 | W | 18.82 | W |
| 19.18 | S | 20.11 | W | 20.86 | M |
| 21.85 | S | 22.50 | W | 22.81 | M |
| 22.96 | M | 24.43 | W | 24.72 | M |
| 25.15 | M | 25.41 | M | 25.63 | M |
| 26.44 | W | 26.65 | W | 27.31 | S |
| 28.00 | W | 29.30 | W | 29.66 | W |
| 34.45 | W | 34.66 | W | 36.83 | W |
| 37.92 | M | 38.35 | W | 44.17 | W |
| 44.71 | W | 47.94 | W | 48.21 | W |

In another preferred example, the X-ray powder diffraction pattern of crystal form IV is substantially as shown in FIG. 22.

In another preferred example, the polymorph is crystal form V of the compound of formula X, the X-ray powder diffraction pattern of which has peaks at diffraction angles 2θ(°) values of the following group V-1: 7.00±0.20, 8.98±0.20, 9.55±0.20, 11.11±0.20, 12.46±0.20, 13.42±0.20, 14.68±0.20, 15.25±0.20, 15.45±0.20, 17.59±0.20, 19.30±0.20, 20.86±0.20, 21.88±0.20, 22.99±0.20, 27.49±0.20, 37.90±0.20.

In another preferred example, the X-ray powder diffraction pattern of the crystal form V further has peaks at 2 or more than 2 of diffraction angles 2θ (°) values selected from the following group V-2: 14.05±0.20, 16.15±0.20, 16.54±0.20, 16.90±0.20, 18.06±0.20, 18.49±0.20, 20.05±0.20, 20.20±0.20, 22.06±0.20, 23.23±0.20, 24.55±0.20, 24.79±0.20, 25.36±0.20, 25.51±0.20, 25.68±0.20, 27.34±0.20, 28.12±0.20.

In another preferred example, the X-ray powder diffraction pattern of the crystal form V further has peaks at 2 or more than 2 of diffraction angles 2θ (°) values selected from the following group V-3: 1.06±0.20, 11.41±0.20, 22.51±0.20, 29.09±0.20, 34.31±0.20, 34.70±0.20, 36.85±0.20, 38.29±0.20, 44.05±0.20, 44.61±0.20.

In another preferred example, the X-ray powder diffraction pattern of the crystal form V has peaks at 6 or more or all (such as 6, 7, 8, 9, 10, 11, 12, 13, 14, 15, etc.) of 2θ (°) values selected from the groups V-1, V-2 and V-3.

In another preferred example, the X-ray powder diffraction pattern of the crystal form V has peaks at 2θ(°) values shown in table V1, and the relative intensity of each peak is as shown in table V1:

TABLE V1

| 2θ(°) | I/I₀ | 2θ(°) | I/I₀ | 2θ(°) | I/I₀ |
|---|---|---|---|---|---|
| 1.06 | W | 7.00 | VS | 8.98 | S |
| 9.55 | S | 11.11 | S | 11.41 | W |
| 12.46 | VS | 13.42 | VS | 14.05 | M |
| 14.68 | VS | 15.25 | S | 15.45 | S |
| 16.15 | M | 16.54 | M | 16.90 | M |
| 17.59 | S | 18.06 | M | 18.49 | M |
| 19.30 | S | 20.05 | M | 20.20 | M |
| 20.86 | S | 21.88 | S | 22.06 | M |
| 22.51 | W | 22.99 | S | 23.23 | M |
| 24.55 | M | 24.79 | M | 25.36 | M |
| 25.51 | M | 25.68 | M | 27.34 | M |
| 27.49 | S | 28.12 | M | 29.09 | W |
| 34.31 | W | 34.70 | W | 36.85 | W |
| 37.90 | S | 38.29 | W | 44.05 | W |
| 44.61 | W | | | | |

In another preferred example, the X-ray powder diffraction pattern of crystal form V is substantially as shown in FIG. 24.

In another preferred example, the crystal form V has an exothermic peak at 238.92° C. (see FIG. 25).

In the second aspect of the invention, there is provided a process for preparing the pharmaceutically acceptable salt of the compound of formula X, or the polymorph of the compound of formula X or its pharmaceutically acceptable salt according to the first aspect of the present invention, comprising steps:

(1) compound 5a is reacted with compound 1a in a solvent thereby to form the compound of formula X; and

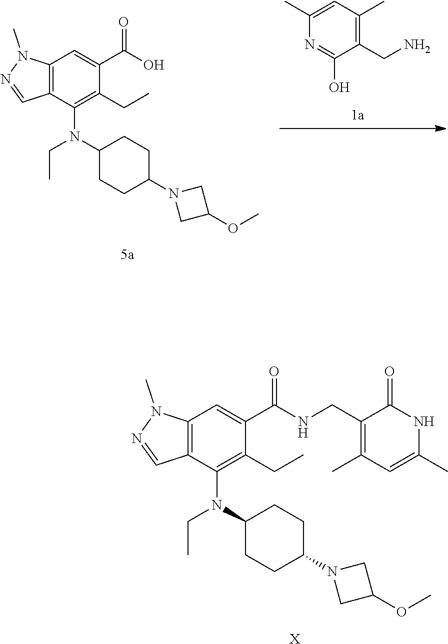

(2) optionally, the compound of formula X and an acid conduct a salt-forming reaction thereby to form a pharmaceutically acceptable salt;

(3) the compound of formula X formed in step (1), or a pharmaceutically acceptable salt thereof formed in step (2) is optionally crystallized thereby to obtain a polymorph.

In another preferred example, the process comprises any one of the following sub-processes (A)-(G) and (I)-(V):

(A) the polymorph is Form A crystal of the hydrochloride of compound of formula X, i.e. crystal form A, and the step (3) comprises crystallization process of the compound of formula X in a solvent in the presence of hydrochloric acid, thereby forming the crystal form A.

In another preferred example, in step (A), the solvent is selected from the group consisting of methanol, ethanol, propanol, isopropanol, butanol, acetone, acetonitrile, tetrahydrofuran, ethyl acetate, propylene glycol, or a mixture thereof, preferably, the organic solvent is tetrahydrofuran or ethyl acetate.

In another preferred example, in step (A), the molar ratio of hydrochloric acid to the compound of formula X is (0.8-2.1):1, preferably (0.9-1.1):1.

In another preferred example, in step (A), the crystallization process is slow cooling.

In another preferred example, in step (A), the temperature for crystallization process is 0-60° C., preferably 4-50° C.

In another preferred example, in step (A), the time for crystallization process is 1-72 hours, preferably 10-50 hours.

(B) the polymorph is Form B crystal of the maleate of compound of formula X, i.e. crystal form B, and the step (3) comprises crystallization process of the compound of formula X in a solvent in the presence of maleic acid, thereby forming the crystal form B.

In another preferred example, in step (B), the solvent is selected from the group consisting of methanol, ethanol, propanol, isopropanol, butanol, acetone, acetonitrile, tetrahydrofuran, ethyl acetate, propylene glycol, or a mixture thereof, preferably, the organic solvent is ethanol, tetrahydrofuran, ethyl acetate or acetone.

In another preferred example, in step (B), the molar ratio of maleic acid to the compound of formula X is (0.8-1.2): 1, preferably (0.9-1.1):1, more preferably 1:1.

In another preferred example, in step (B), the crystallization process is slow cooling or anti-solvent addition.

In another preferred example, in step (B), the temperature for crystallization process is 0-60° C., preferably 4-50° C.

In another preferred example, in step (B), the time for crystallization process is 1-72 hours, preferably 10-50 hours.

(C) the polymorph is Form C crystal of the sulfate of compound of formula X, i.e. crystal form C, and the step (3) comprises crystallization process of the compound of formula X in a solvent in the presence of sulfuric acid, thereby forming the crystal form C.

In another preferred example, in step (C), the solvent is selected from the group consisting of methanol, ethanol, propanol, isopropanol, butanol, acetone, acetonitrile, tetrahydrofuran, ethyl acetate, propylene glycol, or a mixture thereof, preferably, the organic solvent is acetone.

In another preferred example, in step (C), the crystallization process is slow cooling.

In another preferred example, in step (C), the temperature for crystallization process is 0-60° C., preferably 4-50° C.

In another preferred example, in step (C), the time for crystallization process is 1-72 hours, preferably 10-50 hours.

(D) the polymorph is Form D crystal of the hydrobromide of compound of formula X, i.e. crystal form D, and the step (3) comprises crystallization process of the compound of formula X in a solvent in the presence of hydrobromic acid, thereby forming the crystal form D.

In another preferred example, in step (D), the solvent is selected from the group consisting of methanol, ethanol, propanol, isopropanol, butanol, acetone, acetonitrile, tetrahydrofuran, ethyl acetate, propylene glycol, or a mixture thereof, preferably, the organic solvent is tetrahydrofuran or ethyl acetate.

In another preferred example, in step (D), the crystallization process is slow cooling.

In another preferred example, in step (D), the temperature for crystallization process is 0-60° C., preferably 4-50° C.

In another preferred example, in step (D), the time for crystallization process is 1-72 hours, preferably 10-50 hours.

(E) the polymorph is Form E crystal of the methanesulfonate of compound of formula X, i.e. crystal form E, and the step (3) comprises crystallization process of the compound of formula X in a solvent in the presence of methanesulfonic acid, thereby forming the crystal form E.

In another preferred example, in step (E), the solvent is selected from the group consisting of methanol, ethanol, propanol, isopropanol, butanol, acetone, acetonitrile, tetrahydrofuran, ethyl acetate, propylene glycol, or a mixture thereof, preferably, the organic solvent is acetone or ethyl acetate.

In another preferred example, in step (E), the crystallization process is slow cooling.

In another preferred example, in step (E), the temperature for crystallization process is 0-60° C., preferably 4-50° C.

In another preferred example, in step (E), the time for crystallization process is 1-72 hours, preferably 10-50 hours.

(F) the polymorph is Form F crystal of the L-tartrate of compound of formula X, i.e. crystal form F, and the step (3) comprises crystallization process of the compound of formula X in a solvent in the presence of L-tartaric acid, thereby forming the crystal form F.

In another preferred example, in step (F), the solvent is selected from the group consisting of methanol, ethanol, propanol, isopropanol, butanol, acetone, acetonitrile, tetrahydrofuran, ethyl acetate, propylene glycol, or a mixture thereof, preferably, the organic solvent is ethyl acetate.

In another preferred example, in step (F), the crystallization process is slow cooling.

In another preferred example, in step (F), the temperature for crystallization process is 0-60° C., preferably 4-50° C.

In another preferred example, in step (F), the time for crystallization process is 1-72 hours, preferably 10-50 hours.

(G) the polymorph is Form G crystal of the citrate of compound of formula X, i.e. crystal form G, and the step (3) comprises crystallization process of the compound of formula X in a solvent in the presence of citric acid, thereby forming the crystal form G.

In another preferred example, in step (G), the solvent is selected from the group consisting of methanol, ethanol, propanol, isopropanol, butanol, acetone, acetonitrile, tetrahydrofuran, ethyl acetate, propylene glycol, or a mixture thereof, preferably, the organic solvent is acetone or ethyl acetate.

In another preferred example, in step (G), the crystallization process is slow cooling.

In another preferred example, in step (G), the temperature for crystallization process is 0-60° C., preferably 4-50° C.

In another preferred example, in step (G), the time for crystallization process is 1-72 hours, preferably 10-50 hours.

(I) the polymorph is crystal form I of the compound of formula X, and the step (3) comprises crystallization process of the compound of formula X in a solvent, thereby forming the crystal form I.

In another preferred example, in step (I), the solvent is selected from the group consisting of water, methanol, ethanol, propanol, isopropanol, butanol, acetone, acetonitrile, tetrahydrofuran, acetonitrile, propylene glycol, ethyl acetate, methyl isobutyl ketone, isopropyl acetate, 2-methyltetrahydrofuran, dichloromethane, methyl tert-butyl ether, dimethyl sulfoxide, toluene, N,N-dimethylacetamide, N-methylpyrrolidone, or a combination thereof.

In another preferred example, in step (I), the crystallization process is slow volatilization, slow cooling or suspension shaking.

In another preferred example, in step (I), the temperature for crystallization process is 0-60° C.

In another preferred example, in step (I), the time for crystallization process is 2 hours-10 days.

(II) the polymorph is crystal form II of the compound of formula X, and the step (3) comprises crystallization process of the compound of formula X in a solvent, thereby forming the crystal form II.

In another preferred example, in step (II), the solvent is selected from the group consisting of water, methanol, ethanol, propanol, isopropanol, butanol, acetone, acetonitrile, tetrahydrofuran, propylene glycol, ethyl acetate, methyl isobutyl ketone, isopropyl acetate, 2-methyltetrahydrofuran, dichloromethane, methyl tert-butyl ether, dimethyl sulfoxide, toluene, N,N-dimethylacetamide, N-methylpyrrolidone, or a combination thereof. Preferably a mixture of water and acetonitrile.

In another preferred example, in step (II), the crystallization process is slow volatilization or suspension shaking.

In another preferred example, in step (II), the temperature for crystallization process is 0-60° C.

In another preferred example, in step (II), the time for crystallization process is 2 hours-10 days.

(III) the polymorph is crystal form III of the compound of formula X, and the step (3) comprises crystallization process of the compound of formula X in a solvent, thereby forming the crystal form III.

In another preferred example, in step (III), the solvent is selected from the group consisting of water, methanol, ethanol, propanol, isopropanol, butanol, acetone, acetonitrile, tetrahydrofuran, propylene glycol, ethyl acetate, methyl isobutyl ketone, isopropyl acetate, 2-methyltetrahydrofuran, dichloromethane, methyl tert-butyl ether, dimethyl sulfoxide, toluene, N,N-dimethylacetamide, N-methylpyrrolidone, or a combination thereof. Preferably ethanol or isopropanol.

In another preferred example, in step (III), the crystallization process is slow volatilization or suspension shaking.

In another preferred example, in step (III), the temperature for crystallization process is 0-60° C.

In another preferred example, in step (III), the time for crystallization process is 2 hours-10 days.

(IV) the polymorph is crystal form IV of the compound of formula X, and the step (3) comprises crystallization process of the mixture of crystal form I, crystal form II and crystal form III of the compound of formula X in a solvent, thereby forming the crystal form IV.

In another preferred example, in step (IV), the solvent is selected from the group consisting of water, methanol, ethanol, propanol, isopropanol, butanol, acetone, acetonitrile, tetrahydrofuran, propylene glycol, ethyl acetate, 1,4-dioxane, 2-methyltetrahydrofuran, or a mixture thereof. Preferably water.

In another preferred example, in step (IV), the crystallization process is mixing shaking.

In another preferred example, in step (IV), the temperature for crystallization process is 0-60° C.

In another preferred example, in step (IV), the time for crystallization process is 2 hours-10 days.

(V) the polymorph is crystal form V of the compound of formula X, and the step (3) comprises crystallization process of the mixture of crystal form I, crystal form II and crystal form III of the compound of formula X in a solvent, thereby forming the crystal form V.

In another preferred example, in step (V), the solvent is selected from the group consisting of methanol, ethanol, propanol, isopropanol, butanol, acetone, acetonitrile, tetrahydrofuran, propylene glycol, ethyl acetate, 1,4-dioxane, 2-methyltetrahydrofuran, or a mixture thereof. Preferably acetone, ethyl acetate, tetrahydrofuran, acetonitrile or methyl tert-butyl ether.

In another preferred example, in step (V), the crystallization process is mixing shaking.

In another preferred example, in step (V), the temperature for crystallization process is 0-60° C.

In another preferred example, in step (V), the time for crystallization process is 2 hours-10 days.

In the third aspect of the invention, there is provided a pharmaceutical composition comprising:

(a) the pharmaceutically acceptable salt of the compound of formula X, or the polymorph of the compound of formula X or its pharmaceutically acceptable salt according to any one of the first aspect of the present invention; and (b) a pharmaceutically acceptable carrier.

In the fourth aspect of the invention, there is provided a use of the pharmaceutically acceptable salt of the compound of formula X, or the polymorph of the compound of formula X or its pharmaceutically acceptable salt according to the first aspect of the present invention, or the pharmaceutical composition according to the third aspect of the present invention in the preparation of EZH2 inhibitors.

In the fifth aspect of the invention, there is provided a use of the pharmaceutically acceptable salt of the compound of formula X, or the polymorph of the compound of formula X or its pharmaceutically acceptable salt according to the first aspect of the present invention, or the pharmaceutical composition according to the third aspect of the present invention in the preparation of drugs for EZH2-mediated diseases or conditions.

In the sixth aspect of the present invention, there is provided a method of treating a disease or condition mediated by EZH2, comprising administering to a patient in need thereof a therapeutically effective amount of the pharmaceutically acceptable salt of the compound of formula X, or the polymorph of the compound of formula X or its pharmaceutically acceptable salt according to the first aspect of the present invention, or the pharmaceutical composition according to the third aspect of the present invention.

In the seventh aspect of the present invention, there is provided a method of treating a disease or condition mediated by EZH2, comprising administering to a patient in need thereof a therapeutically effective amount of the pharmaceutically acceptable salt of the compound of formula X, or the polymorph of the compound of formula X or its pharmaceutically acceptable salt according to the first aspect of the present invention, and another therapeutically active agent.

In another preferred example, the disease or condition mediated by EZH2 is selected from: cancer, pulmonary arterial hypertension, myelofibrosis, human immunodeficiency virus (HIV) disease, graft versus host disease (GVHD), Weaver syndrome, psoriasis vulgaris or liver fibrosis.

In another preferred example, the disease or condition mediated by EZH2 is cancer.

In another preferred example, the cancer mediated by EZH2 includes, but are not limited to, thyroid cancer, cardiac sarcoma, lung cancer, gastrointestinal cancer, genitourinary tract tumor, liver cancer, mantle cell lymphoma, osteosarcoma, nervous system sarcoma, gynecological cancer, hematological system tumor, adrenal neuroblastoma, skin cancer, astrocytic tumor, breast cancer, colorectal cancer, endometrial cancer, head and neck cancer, oral cavity cancer.

It should be understood that each of the above technical features of the invention and each technical feature specifically described below (such as in Examples) can be combined with each other within the scope of the present invention so as to constitute new or preferred technical solutions which need not be specified again herein.

### BRIEF DESCRIPTION OF THE DRAWINGS

FIG. 1 shows an XRPD pattern of crystal form A.

FIG. 2 shows a DSC pattern of crystal form A.

FIG. 3 shows a TGA pattern of crystal form A.

FIG. 4 shows an XRPD pattern of crystal form B.

FIG. 5 shows a DSC pattern of crystal form B.

FIG. 6 shows a TGA pattern of crystal form B.

FIG. 7 shows an XRPD pattern of crystal form C.

FIG. 8 shows an XRPD pattern of crystal form D.

FIG. 9 shows an XRPD pattern of crystal form E.

FIG. 10 shows an XRPD pattern of crystal form F.

FIG. 11 shows an XRPD pattern of crystal form G.

FIG. 12 shows an XRPD pattern of crystal form I.

FIG. 13 shows a DSC pattern of crystal form I.

FIG. 14 shows a TGA pattern of crystal form I.

FIG. 15 shows a microscroph of crystal form I.

FIG. 16 shows an XRPD pattern of crystal form II.

FIG. 17 shows a DSC pattern of crystal form II.

FIG. 18 shows a microscroph of crystal form II.

FIG. 19 shows an XRPD pattern of crystal form III.

FIG. 20 shows a DSC pattern of crystal form III.

FIG. 21 shows a microscroph of crystal form III.

FIG. 22 shows an XRPD pattern of crystal form IV.

FIG. 23 shows a microscroph of crystal form IV.

FIG. 24 shows an XRPD pattern of crystal form V.

FIG. 25 shows a DSC pattern of crystal form V.

FIG. 26 shows a microscroph of crystal form V.

### DETAILED DESCRIPTION OF THE INVENTION

The inventors have conducted extensive and intensive studies and have unexpectedly found that such 4,5,6-trisubstituted indazole derivatives, particularly the 4,5,6-trisubstituted indazole derivatives, the 7-position of indazole of which is unsubstituted, have high inhibitory activities against enzymes such as EZH2 Y641F and SU-DHL-6 and SU-DHL-10 cells. The study also found that a series of polymorphs of free base, salts and the polymorphs of the salts of the compound of formula X not only had good physical and chemical stability, but also had good pharmacological activity in vivo and in vitro, and therefore could be further developed into drugs.

### Terms

As used herein, “the crystal of the present invention”, “the crystal form of the present invention”, “the polymorph of the present invention”, etc. can be used interchangeably.

Compound of Formula X

In the present invention, the compound of formula X is N-((4,6-dimethyl-2-oxo-1,2-dihydropyridin-3-yl)methyl)-5-ethyl-4-(ethyl((1S,4S)-4-(3-methoxyazetidin-1-yl)cyclohex yl)amino)-1-methyl-1H-indazole-6-carboxamide, which has a high inhibitory activities against enzymes such as EZH2 Y641F and cells such as SU-DHL-6 and SU-DHL-10.

The invention also includes a pharmaceutically acceptable salt of the compound of formula X, or a polymorph of the free base of compound of formula X or its pharmaceutically acceptable salt.

In the present invention, the pharmaceutically acceptable salt is selected from the group consisting of hydrochloride, sulfate, phosphate, maleate, fumarate, L-tartrate, citrate, methanesulfonate and hydrobromide.

Polymorphs

Solid exists in amorphous form or in crystalline form. In the case of crystal form, the molecules are localized in the three-dimensional lattice sites. When a compound is crystallized from a solution or slurry, it can be crystallized in different space lattice arrangement (this property is called “polymorphism”) to form crystals with different crystalline forms, each of which is called as “polymorphs.” Different polymorphs of a given substance may differ from each other in one or more physical properties (such as solubility and dissolution rate, true specific gravity, crystalline form, packing pattern, flowability and/or solid state stability).

Crystallization

Crystallization on the production scale can be accomplished by manipulating a solution such that the solubility limit of the interested compound is exceeded. This can be done in a number of ways, for example, by dissolving the compound at a relatively high temperature and then cooling the solution below a saturation limit, or by boiling, evaporating at ordinary pressure, drying under vacuum or by some other means to reduce the liquid volume. The solubility of the interested compound may be reduced by adding an anti-solvent or a solvent in which the compound has a low solubility or a mixture of such solvents. An alternative method is to adjust the pH to reduce the solubility. See Crystallization, Third Edition, J W Mullens, Butterworth-Heineman Ltd., 1993, ISBN 0750611294 for a detailed description of crystallization.

The “suspension stirring” described in the present invention means a way to get crystals by mixing the compound of formula X with the corresponding acid or a solution of the corresponding acid to form a turbid solution, or by mixing the compound of formula X with a suitable solvent to form a turbid solution before stirring. Suitable solvents may be water or organic solvents.

The “slow volatilization” described in the present invention means a way to get crystals by placing a solution of the compound of formula X or a solution of the compound of formula X and the corresponding acid at a certain temperature for slow volatilization of the solvent.

The “addition of anti-solvent” described in the present invention means a way to get crystals by adding a suitable solvent to a solution of the compound of formula X and precipitating the crystals.

If salt formation and crystallization are expected to occur at the same time, the addition of an appropriate acid or base can result in the direct crystallization of the desired salt if the salt is less soluble in the reaction medium than the raw material. Likewise, in a medium in which the solubility of the desired final form is lower than that of reactant, the final product can be directly crystallized when the synthetic reaction is completed.

Optimization of crystallization can include inoculation of the crystal of desired form as a seed into the crystallization medium. In addition, many crystallization methods include a combination of the above strategies. One example is to dissolve the interested compound in a solvent at a high temperature, followed by the addition of an antisolvent with a suitable volume in a controlled manner so that the system is just below saturation level. At this moment, the seed of desired form (the integrity of the seed is kept) can be added and the system is cooled to complete the crystallization.

As used herein, the term “room temperature” generally means 4-30° C., preferably 20±5° C.

Polymorphs of the Present Invention

As used herein, the term “the polymorph of the present invention” includes the polymorph of the compound of formula X or its pharmaceutically acceptable salts (eg hydrochloride, maleate), or the polymorph of various solvates of the compound of formula X, and also includes different polymorphs of the same salt or solvate.

“the polymorph of the compound of formula X” and “the polymorph of the free base of the compound of formula X” can be used interchangeably.

Preferred polymorphs of the invention include (but are not limited to):
(i) crystal forms A, B, C, D, E, F, G (crystal form of salt);
(ii) crystal forms I, II, III, IV, V (crystal form of the free base of compound of formula X).

In the present invention, some crystal forms can be converted into each other, so the present invention also provides a method of converting some crystal forms into each other.

Identification and Properties of Polymorphs

The properties of the polymorph of the invention were studied using the following various ways and instruments after its preparation.

X-Ray Powder Diffraction

Methods of determining X-ray powder diffraction of the crystals are known in the art. For example, an X-ray powder diffractometer was used to obtain a pattern with a copper radiation target at a scanning speed of 2° per minute.

The polymorph of the compound of formula X of the present invention or a pharmaceutically acceptable salt thereof has a specifiform C crystal and has specific characteristic peaks in an X-ray powder diffraction (XRPD) pattern.

Differential Scanning Calorimetry

It is also called “differential scanning calorimetry analysis” (DSC) which is a technique that measures the relationship between energy difference of the measured substance and the reference substance and temperature during heating. The location, shape and number of peaks on the DSC pattern are related to the nature of the substance, and therefore can be used to qualitatively identify the substance. This method can be commonly used in the art to detect the phase transition temperature, glass transition temperature, reaction heat and other parameters of a substance.

Pharmaceutical Compositions of Compound of Formula X and their Use

Generally, the polymorph of the compound of formula X of the present invention or its pharmaceutically acceptable salt may form a suitable dosage form for administration with one or more pharmaceutically acceptable carriers. These dosage forms are suitable for oral, rectal, topical, intraoral administration, and other parenteral administration (e.g., subcutaneous, intramuscular, intravenous administration, etc.). For example, dosage forms suitable for oral administration include capsules, tablets, granules and syrups. Compounds of the present invention contained in these formulations may be solid powders or granules; aqueous or non-aqueous liquid solutions or suspensions; water-in-oil or oil-in-water emulsions. Such dosage forms may be prepared with active compounds and one or more carriers or excipients through the conventional pharmacy methods. The above-mentioned carriers should be compatible with active compounds or other excipients. For solid formulations, conventional non-toxic carriers include, but not limited to mannitol, lactose, starch, magnesium stearate, cellulose, glucose, sucrose and the like. Carriers used for liquid preparations include water, saline, aqueous dextrose, ethylene glycol and polyethylene glycol. The active compounds may form a solution or suspension with the above-mentioned carriers.

The compositions of the present invention are formulated, quantified and administrated in a manner consistent with the practice of medicine. The “effective amount” of the administrated compound depends on the factors such as the specific disease to be treated, the individual being treated, the cause of diseases, the drug targets and the mode of administration, etc.

The present invention provides the uses of a pharmaceutically acceptable salt of the compound of formula X, or a polymorph of the compound of formula X or its pharmaceutically acceptable salt according to the first aspect of the present invention in the preparation of an EZH2 inhibitor or a medicament for treating EZH2-mediated diseases or conditions.

In another preferred example, the disease or condition mediated by EZH2 is selected from: cancer, pulmonary arterial hypertension, myelofibrosis, human immunodeficiency virus (HIV) disease, graft versus host disease (GVHD), Weaver syndrome, psoriasis vulgaris or liver fibrosis.

In another preferred example, the disease or condition mediated by EZH2 is cancer.

In another preferred example, the cancer mediated by EZH2 includes, but are not limited to, thyroid cancer, cardiac sarcoma, lung cancer, gastrointestinal cancer, genitourinary tract tumor, liver cancer, mantle cell lymphoma, osteosarcoma, nervous system sarcoma, gynecological cancer, hematological system tumor, adrenal neuroblastoma, skin cancer, astrocytic tumor, breast cancer, colorectal cancer, endometrial cancer, head and neck cancer, oral cavity cancer.

As used herein, “therapeutically effective amount” refers to the amount that yields a function or activity to humans and/or animals and may be tolerated by humans and/or animals.

The therapeutically effective amount of the pharmaceutical composition of the present invention or the pharmaceutically acceptable salt of the compound of formula X, or the polymorph of the compound of formula X or its pharmaceutically acceptable salt contained in the pharmaceutical composition is preferably 0.1 mg-5 g/kg (body weight).

The Main Advantages of the Present Invention are:

The inventor found that the polymorphs of N-((4,6-dimethyl-2-oxo-1,2-dihydropyridin-3-yl)methyl)-5-ethyl-4-(ethyl((1S,4S)-4-(3-methoxyazetidin-1-yl)cyclohexyl)amino)-1-methyl-1H-indazole-6-carboxamide free base and salts thereof also had good physical and chemical stability and outstanding related pharmacological activities.

### DETAILED DESCRIPTION OF THE INVENTION

The present invention will be further illustrated below with reference to the specific examples. It should be understood that these examples are only to illustrate the invention but not to limit the disclosure of the invention. The experimental methods without specific conditions in the following embodiments are generally carried out according to conventional conditions, or in accordance with the conditions recommended by the manufacturer. Unless indicated otherwise, parts and percentage are calculated by weight.

Reagents and Instruments

In the present invention, the structure and purity of the compounds are identified by nuclear magnetic resonance (¹HNMR) and, or LC-MS mass spectrometry (LC-MS). ¹HNMR: BrukerAVANCE-400 NMR machine, the internal standard was tetramethylsilane (TMS). LC-MS: Agilent 1200 HPLC System, 6140 MS liquid-mass spectrometer (available from Agilent), column WatersX-Bridge, 150×4.6 mm, 3.5 μm. Preparative high performance liquid chromatography (pre-HPLC): Waters PHW007, column XBridge C18, 4.6*150 mm, 3.5 um.

ISCO Combiflash-Rf75 or Rf200 automatic eluting column instrument, Agela 4 g, 12 g, 20 g, 40 g, 80 g, 120 g disposable silica gel column.

Thin-layer silica gel plate was Yantai Huanghai HSGF254 or Qingdao GF254 silica gel plates, thin layer chromatography (TLC), silica gel plates used for detection of reaction are 0.15 mm-0.2 mm, silica gel plates using for TLC purification of products are 0.4 mm-0.5 mm. Yantai Huanghai 200-300 mesh silica gel is generally used as a carrier. FCP200-300 mesh alkaline aluminum oxide is commonly used as a carrier in alkaline aluminum oxide column.

All examples were performed under nitrogen or argon atmosphere and the solution refers to the aqueous solution if without special explanation.

As used herein, DMF refers to dimethylformamide, DMSO refers to dimethylsulfoxide, THF refers to tetrahydrofuran, DIEA refers to N,N-diisopropylethylamine, EA refers to ethyl acetate, PE refers to petroleum ether, BINAP refers to (2R,3S)-2,2′-bis diphenylphosphino-1,1′-binaphthyl, NBS refers to N-bromosuccinimide, NCS refers to N-chlorosuccinimide, Pd₂(dba)₃ refers to tris(dibenzylideneacetone)dipalladium, and Pd(dppf)Cl₂ refers to [1,1′-bis (diphenylphosphino) ferrocene]palladium dichloride.

Acetonitrile ACN, methanol MeOH, ethanol EtOH, isopropyl alcohol IPA, acetone ACE, ethyl acetate EA, methyl tert-butyl ether MTBE, tetrahydrofuran THF, water H₂O, 50% acetonitrile 50% ACN.

As used herein, room temperature refers to about 20±5° C.

General Method

X-ray powder diffraction: In the present invention, the powder X-ray diffraction patterns of the above crystal forms are obtained by a method known in the art, using a PANalytacal Empyrean X-ray powder diffraction analyzer. The instrument test conditions are shown in the following table:

| Parameter | XRPD(Reflection mode) |
|---|---|
| X-ray | Cu, kα, Kα1 (Å): 1.540598; |
| | Kα2 (Å): 1.544426 |
| | Ka2/Ka1 Intensity ratio: 0.50 |
| X-ray tube settings | 45 kV, 40 mA |
| divergence slit | automatic |
| monochromator | none |
| Scan mode | continuous |
| Scanning range (°2Theta) | 3°~40° |
| Scanning step (°2Theta) | 0.013 |
| Scan time (minutes) | ~7 |

In the powder X-ray diffraction pattern, the site of each peak was determined by 2θ(°). It should be understood that different instruments and/or conditions could result in slightly different data and changes in peak site and relative intensity. The division of the intensity of peaks only reflects the approximate size of peaks in each site. In the present invention, the highest diffraction peak of each crystalline form was taken as the base peak which was defined as I₀ with the relative intensity as 100%, (the peak of crystal form I with 2θ(°) value of 7.09 is the base peak, the peak of crystal form II with 2θ(°) value of 7.112 is the base peak, the peak of crystal form III with 2θ(°) value of 6.969 is the base peak, the peak of crystal form IV with 2θ(°) value of 8.978 is the base peak, the peak of crystal form V with 2θ(°) value of 7 is the base peak, the peak of crystal form A with 2θ(°) value of 9.311 is the base peak, the peak of crystal form B with 2θ(°) value of 13.958 is the base peak, the peak of crystal form C with 2θ(°) value of 14.436 is the base peak, the peak of crystal form D with 2θ(°) value of 14.653 is the base peak, the peak of crystal form E with 2θ(°) value of 13.899 is the base peak, the peak of crystal form F with 2θ(°) value of 15.309 is the base peak, the peak of crystal form G with 2θ(°) value of 15.22 is the base peak), and other peaks had the ratio of their peak height to the peak height of base peak as the relative intensity I, I₀. The definition of the relative intensity of each peak was shown in the following table:

| Relative intensity I/I₀(%) | Definition |
|---|---|
| 50~100 | VS (very strong) |
| 25~50 | S (strong) |
| 10~25 | M (medium) |
| 1~10 | W (weak) |

The acid-base molar ratio of the salts of the present invention or their crystalline forms was determined by HPLC/IC or ¹H NMR.

High performance liquid chromatography: In the present invention, high performance liquid chromatography (HPLC) is collected on an Agilent 1100/1260 HPLC.

TGA and DSC pattern: TGA and DSC pattern were collected on TA Q500/5000 thermogravimetric analyzer and TA Q200/2000 differential scanning calorimeter respectively. The instrument test conditions are shown in the following table:

| Parameter | TGA | DSC |
|---|---|---|
| Method | Linear warming | Linear warming |
| Sample tray | Platinum plate, open | Aluminum plate, gland |
| Temperature range | Room temperature - | 25° C. - set temperature |
| | set temperature | |
| Scanning rate (° C., min) | 10 | 10 |
| Protective gas | Nitrogen | Nitrogen |

The Dynamic Vapor Sorption (DVS) curve: acquired on the DVS Intrinsic of Surface Measurement Systems. The relative humidity at 25° C. is corrected with the deliquescence points of LiCl, Mg(NO₃)₂ and KCl. The instrument test conditions are shown in the following table:

| Parameters | Setting value | |
|---|---|---|
| Temperature | 25° | C. |
| Sample size | 10~20 | mg |
| Protect gas and flow | N₂, 200 | ml/min |
| dm/dt | 0.002%/min | |
| Minimum dm/dt balance time | 10 | minutes |
| The maximum balance time | 180 | minutes |

| | |
|---|---|
| RH range | 0% RH-90% RH-0% RH |
| RH gradient | 10%(0% RH-90% RH, 90% RH-0% RH) |
| | 5%(90% RH-95% RH, 5% RH-90% RH) |

Moisture content: tested with Metrohm 870 Karl Fischer Moisture Analyzer, titration test solution used is Hydranal®R-Composite5 (34805-1L-R, Batch #SZBD3330V) commercially available from Sigma-aldrich, and analytically pure MeOH is used as solvent. Calibrate with high-purity water before measuring moisture.

It should be understood that different values may be obtained when other types of instruments with the same function as the instruments described above or test conditions which are different from the conditions used in the present invention were used. Therefore, the recited value should not be considered as an absolute numerical value.

Due to the instrumental errors or different operators, one skilled in the art will understand that the above parameters used to characterize the physical properties of crystals may differ slightly, so the parameters described above are only used to assist in characterizing the polymorphs provided herein, and can not be regarded as a limitation on the polymorphs of the present invention.

Preparation of Intermediates

Preparation of Compound 1a

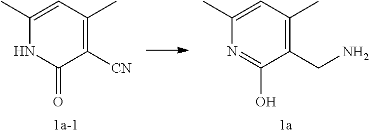

The solution of compound 1a-1 (22.5 g, 152 mmol) in tetrahydrofuran (500 mL) was slowly added lithium aluminum hydride (11.5 g, 0.3 mol) under an ice bath, and the mixture was stirred at room temperature overnight. To the system were added 15 mL water, 30 mL sodium hydroxide solution (15%), filtered, and the filtrate was concentrated to give a white solid compound 1a. MS m/z (ESI): N/A.

Preparation of Compound 2a

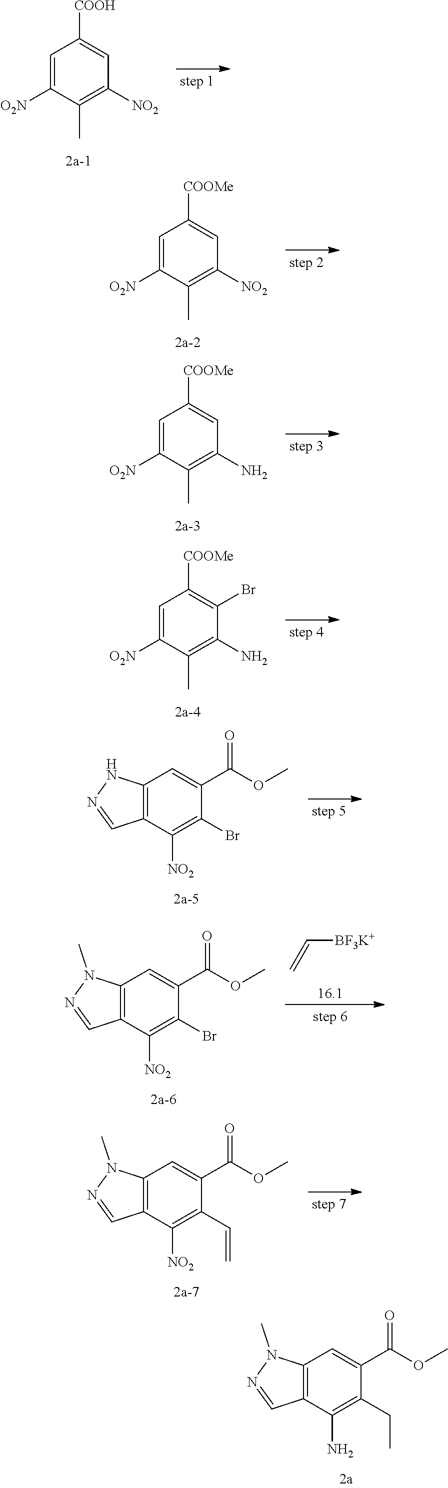

Step 1: A solution of compound 2a-1 (8 g, 35.3 mmol) in anhydrous methanol (100 mL) was added dichlorosulfoxide (7.7 mL, 106.1 mmol), the mixture was stirred at room temperature and then under reflux for 20 hours. LC-MS followed until the reaction was complete. After cooling, the reaction solution was concentrated to remove most of the solvent, filtered, and the filter cake was dried under reduced pressure to obtain 7.5 g of compound 2a-2. MS m/z (ESI): 241[M+H]⁺.

Step 2: A solution of compound 2a-2 (7.6 g, 31.64 mmol) in acetic acid (150 mL) was added iron powder (14 g, 253 mmol) in portions. After addition, the reaction solution was filtered, the filtrate was poured into water, extracted with ethyl acetate, and the organic layer was concentrated, purified by combiflash to give a yellow solid compound 2a-3 (3.5 g, 52.6%). MS m/z (ESI): 211[M+H]⁺.

Step 3: A solution of compound 2a-3 (1 g, 4.76 mmol) in DMSO (20 mL) was added dropwise a solution of NBS (931 mg, 5.23 mmol) in DMSO (1.5 mL), the mixture was stirred at room temperature for 2 hours.

LC-MS followed until the reaction was complete. The reaction solution was poured into water, filtered, and the filter cake was dried under reduced pressure to obtain 700 mg of yellow solid compound 2a-4. MS m/z (ESI): 290.8[M+H]⁺.

Step 4: A solution of compound 2a-4 (2.36 g, 8.16 mmol) in acetic acid (30 mL) was added dropwise a solution of sodium nitrite (619 mg, 8.98 mmol) in water (5 mL) at 40° C., and the mixture was stirred at 40° C. for 1 hour. LC-MS followed until the reaction was complete. The reaction solution was poured into water, extracted with ethyl acetate, dried and concentrated to give 2 g of solid compound 2a-5. MS m/z (ESI): 299.8[M+H]⁺.

Step 5: 5 mL of a solution of compound 2a-5 (88.5 mg, 0.296 mmol) in N,N-dimethylformamide was added sodium hydrogen (24 mg, 0.591 mmol) under an ice bath, and stirred for 30 minutes under an ice bath, iodomethane (84 mg, 0.591 mmol) was added, the mixture was stirred for another 2 hours. LC-MS followed until the reaction was complete. The reaction solution was poured into water, extracted with ethyl acetate, dried and concentrated to give 60 mg of compound 2a-6. MS m/z (ESI): 315.8[M+H]⁺.

Step 6: A mixture of compound 2a-6 (904 mg, 2.87 mmol), compound 16.1 (774 mg, 5.73 mmol), Pd (dppf) C12 (210 mg, 0.286 mmol), sodium carbonate (607 mg, 5.73 mmol), 1,4-dioxane (20 mL) and 2 mL of water was reacted in microwave at 100° C. for 8 hours under an argon atmosphere. LC-MS followed until the reaction was complete. The reaction solution was poured into water, extracted with ethyl acetate, concentrated and purified by combiflash to obtain a yellow solid compound 2a-7 (300 mg, 20%). MS m/z (ESI): 262.1[M+H]⁺.

Step 7: A solution of compound 2a-7 (52.2 mg, 0.2 mmol) in methanol (5 mL) and 1 mL tetrahydrofuran was added palladium carbon (15 mg), the mixture was stirred at room temperature under a hydrogen atmosphere overnight. LC-MS followed until the reaction was complete. The reaction solution was filtered, and concentrated to give compound 2a. MS m/z (ESI): 234.2[M+H]⁺.

### Example 1 Preparation of Compound of Formula X

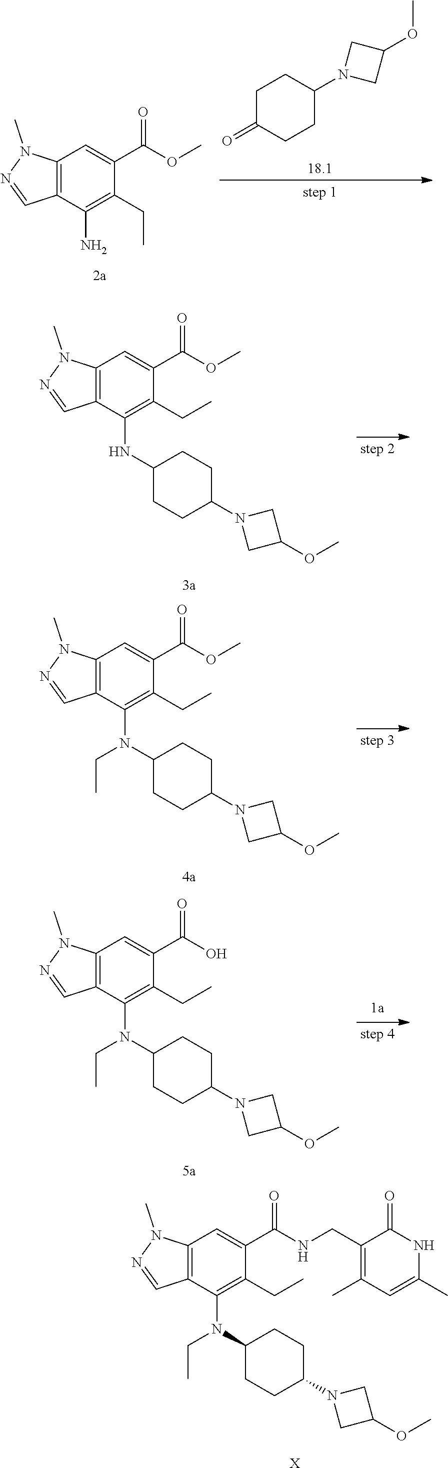

Step 1: A solution of compound 2a (828 mg, 3.54 mmol), compound 18.1 (1.3 g, 7.08 mmol), and 5 mL of trifluoroacetic acid in 1,4-dioxane (50 mL) was stirred at room temperature for 2 hours, sodium triacetoxyborohydride (2.25 mg, 10.62 mmol) was added, then the mixture was stirred at room temperature for 4 hours. LC-MS followed until the reaction was complete. The reaction solution was poured into water, adjusted to pH 8 with sodium bicarbonate solution, extracted with ethyl acetate and dried, concentrated to give 1 g of compound 3a. MS m/z (ESI): 401[M+H]⁺.

Step 2: A solution of compound 3a (1.29 g, 3.22 mmol), acetaldehyde (710 mg, 16.12 mmol), and 3 mL of acetic acid in 1,4-dioxane (30 mL) was stirred at room temperature for 2 hours, sodium triacetoxyborohydride (2.05 g, 9.67 mmol) was added, and the mixture was stirred at room temperature overnight. LC-MS followed until the reaction was complete. The reaction solution was poured into water, adjusted to pH 8 with sodium bicarbonate solution, extracted with ethyl acetate and concentrated, and purified by combiflash to obtain a red compound 4a (280 mg, 26%). MS m/z (ESI): 429.3 [M+H]⁺.

Step 3: A solution of compound 4a (310 mg, 0.724 mmol) in THF (10 mL) was added methanol (4 mL) and sodium hydroxide (4 mL, 3M), the mixture was stirred at room temperature overnight. LC-MS followed until the reaction was complete. The reaction solution was adjusted to pH 6, extracted with ethyl acetate to remove impurity, and the aqueous layer was concentrated. The residue was washed with DCM:MeOH=10:1, the filtrate was concentrated after filtration to obtain 200 mg of compound 5a. MS m/z (ESI): 415 [M+H]⁺.

Step 4: A solution of compound 5a (92 mg, 0.223 mmol) in DMF (3 mL) was added HATU (127 mg, 0.335 mmol), DIPEA (115 mg, 0.892 mmol) and compound 1a (84 mg, 0.446 mmol), the mixture was stirred at room temperature overnight. LC-MS followed until the reaction was complete. The reaction solution was extracted with ethyl acetate/water system, the organic layer was concentrated, and purified by Prep-HPLC to obtain the free base of compound of formula X (15 mg, 13.5%) as a white solid.

¹H NMR (400 MHz, DMSO) δ 11.46 (s, 1H), 8.13 (t, 1H), 8.05 (s, 1H), 7.29 (s, 1H), 5.87 (s, 1H), 4.30 (d, 2H), 3.97 (s, 3H), 3.93-3.89 (m, 1H), 3.43 (s, 2H), 3.13 (s, 3H), 3.08-2.90 (m, 4H), 2.64-2.59 (m, 2H), 2.24 (s, 3H), 2.11 (s, 3H), 2.08 (s, 1H), 1.76-1.43 (m, 6H), 1.20 (s, 3H), 0.97 (t, 3H), 0.79 (t, 3H). MS m/z (ESI): 549[M+H]⁺. The obtained solid was sent for XRD detection, and its powder X-ray diffraction pattern showed no obvious characteristic peaks, so it was in amorphous form.

### Comparative Example

### Comparative Example 1: N-((4,6-dimethyl-2-oxo-1,2-dihydropyridin-3-yl)methyl)-4-(ethyl (tetrahydro-2H-pyran-4-yl) amino)-1,7-dimethyl-1H-indazole-6-carboxamide (D1)

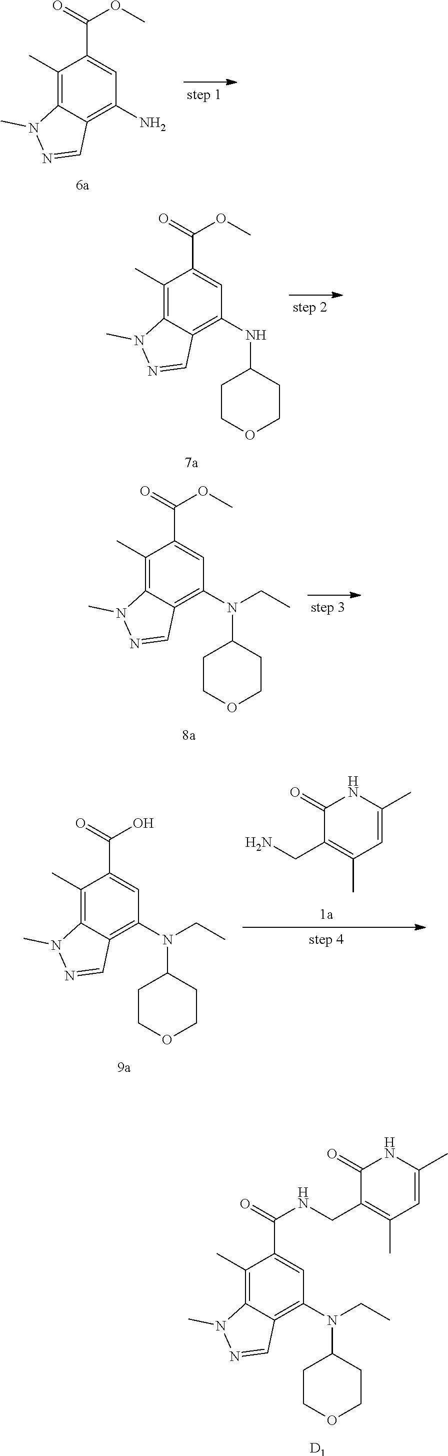

Step 1: A mixed solution of compound 6a (90 mg, 0.408 mmol), tetrahydropyrone (86.6 mg, 0.857 mmol) in dioxane/trifluoroacetic acid (10 ml/2 ml) was stirred at room temperature for 2 h, sodium borohydride acetate (272 mg, 1.29 mmol) was added, the mixture was stirred at room temperature for 1 h, LC-MS followed until the reaction was complete. The reaction was quenched with saturated sodium bicarbonate solution, and the pH was adjusted to 8, extracted with ethyl acetate, the organic phase was dried over anhydrous sodium sulfate, filtered and concentrated to give brown oily compound 7a (150 mg), MS m/z (ESI): 304.2[M+H]⁺.

Step 2: A mixed solution of compound 7a (127 mg, 0.418 mmol) and acetaldehyde (92 mg, 2.07 mmol) in dioxane/acetic acid (20 ml/2 ml) was stirred at room temperature for 1 h, and sodium borohydride acetate was added (443 mg, 2.09 mmol), the mixture was stirred at room temperature for 1 h, LC-MS followed until the reaction was complete. The reaction was quenched with saturated sodium bicarbonate solution, extracted with ethyl acetate, and the organic phase was dried over anhydrous sodium sulfate, filtered and concentrated to give brown oily compound 8a (150 mg), MS m/z (ESI): 332.8[M+H]⁺.

Step 3: To a solution of compound 8a (129 mg, 0.388 mmol) in methanol was added sodium hydroxide solution (4 M, 2 ml), and the mixture was stirred at 50° C. for 5 h, LC-MS followed until the reaction was complete. The reaction solution was concentrated under reduced pressure to remove methanol, diluted with water, extracted with ethyl acetate to remove impurities, the aqueous phase was adjusted to pH of 4 with hydrochloric acid (3 M), extracted with ethyl acetate, the organic phase was dried over anhydrous sodium sulfate, filtered and concentrated to give black oil compound 9a (150 mg), MS m/z (ESI): 318.3[M+H]⁺.

Step 4: To a solution of compound 9a (182.6 mg, 0.576 mmol) in DMF was added compound 1a (83.5 mg, 0.564 mmol), HATU (214 mg, 0.564 mmol) and DIPEA (194 mg, 1.50 mmol), the mixture was stirred at room temperature for 5 h, LC-MS followed until the reaction was complete. The reaction solution was poured into water, extracted with ethyl acetate, the organic phase was washed with brine, dried over anhydrous sodium sulfate, filtered, concentrated and purified by Pre-HPLC to obtain a white solid compound D1 (6 mg, 3%), MS m/z (ESI): 452[M+H]⁺. ¹H NMR (400 MHz, DMSO) δ 11.49 (s, 1H), 8.05 (t, J=4.0 Hz, 1H), 7.88 (s, 1H), 6.36 (s, 1H), 5.87 (s, 1H), 4.28 (d, J=4.0 Hz, 2H), 4.24 (d, J=4.0 Hz, 3H), 3.88-3.85 (m, 2H), 3.64-3.63 (m, 1H), 3.29-3.23 (m, 4H), 2.56 (s, 3H), 2.21 (s, 3H), 2.11 (s, 3H), 1.68-1.66 (m, 4H), 0.96 (t, J=8.0 Hz, 3H).

Comparative compounds D2 to D4 are prepared by referring to the preparation method of comparative compound D1.

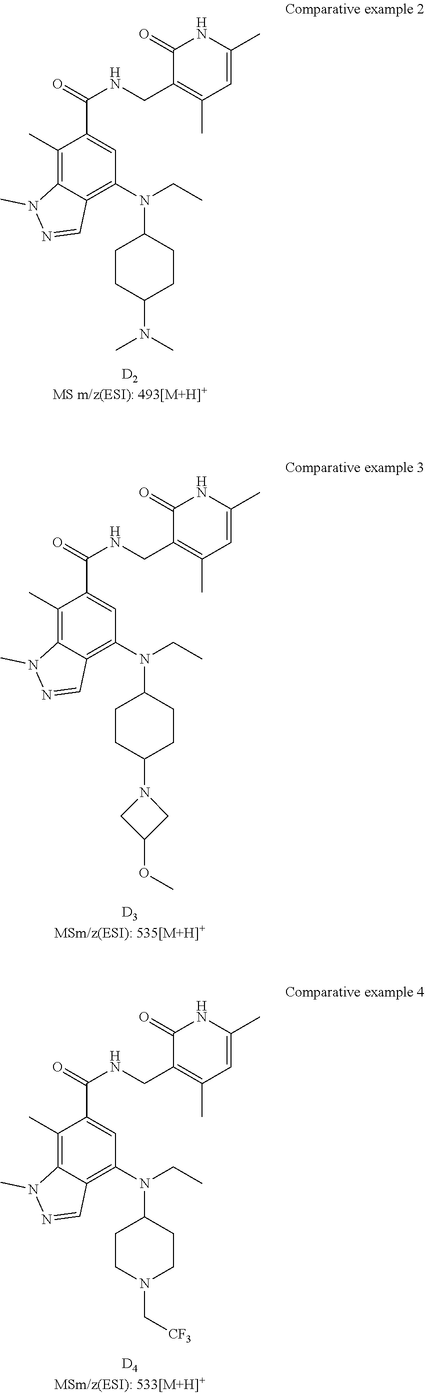

### Example 2 Preparation of Crystal Form A of Compound of Formula X

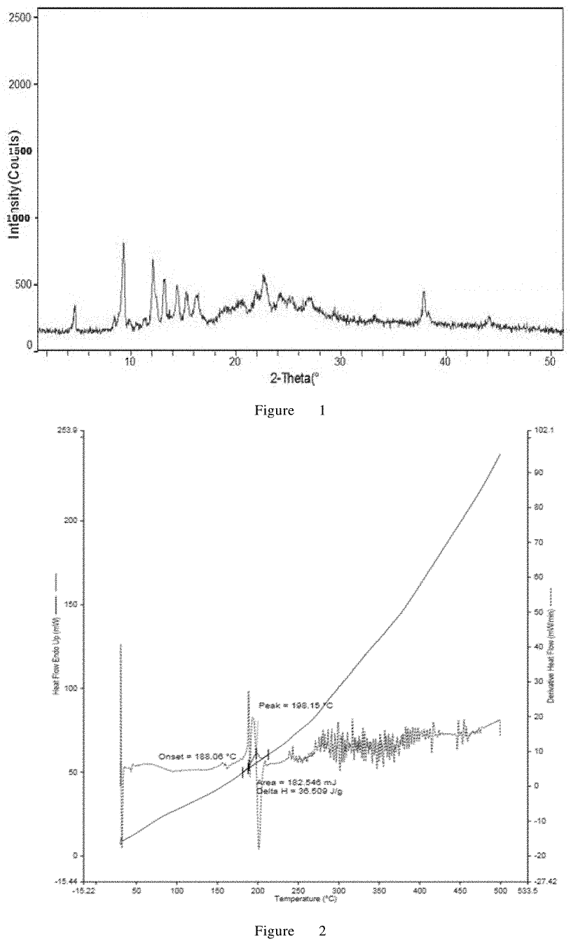

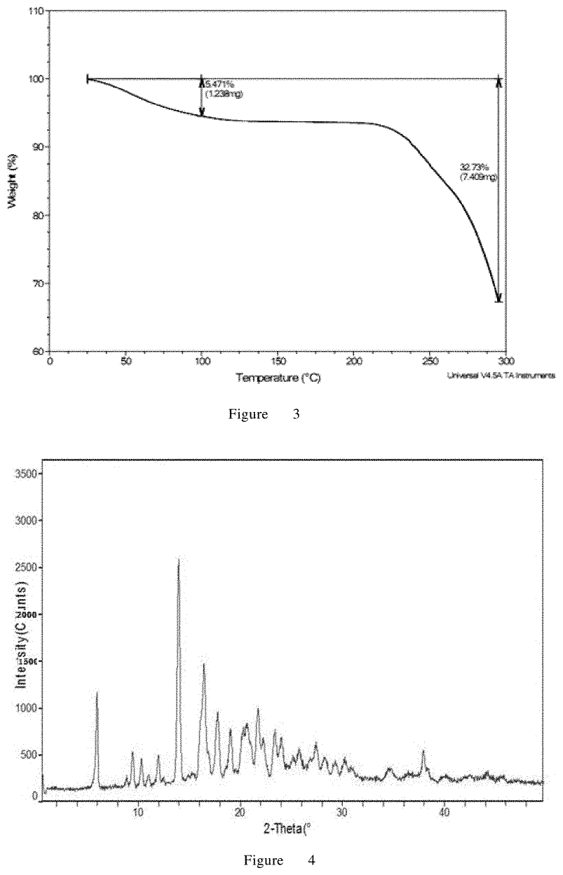

200 mg of the starting sample of free base prepared according to the method of Example 1 was weighed into a 30 ml glass sample bottle, 10 ml of ethyl acetate was added, 440 μL of 1M hydrochloric acid solution was slowly added while stirring at 50° C., reacted at this temperature for 4 h, the solution was a turbid; slowly cooled down to 0° C. after 4 h, solid precipitation increased; the solid was obtained by centrifugal separation, the solvent is evaporated to obtain a solid product. The powder X-ray diffraction pattern of the obtained crystal is shown in FIG. 1 (20 angle has been marked), the crystal form is defined as crystal form A in this application. Its DSC and TGA patterns are shown in FIGS. 2 and 3. The peak temperature of exothermic peak of crystal form A is 198.15° C. TGA results show that the weight loss is 5.471% before 100° C. and the weight loss is 27.259% from 100-295.2° C.

### Example 3 Preparation of Crystal Form B of Compound of Formula X

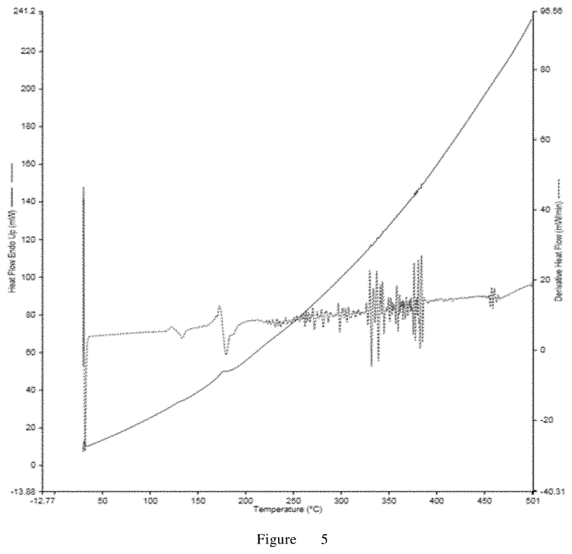

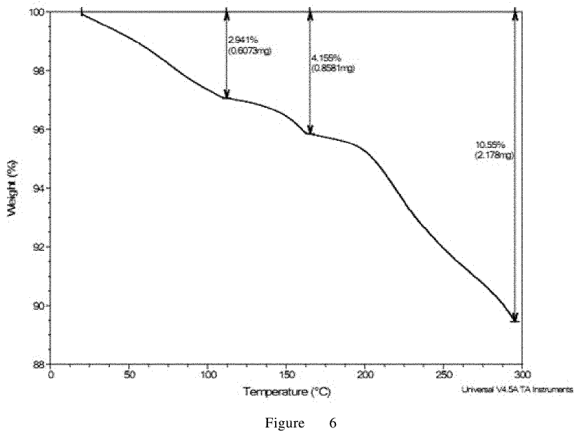

200 mg of the starting sample of free base prepared according to the method of Example 1 was weighed into a 30 ml glass sample bottle, 10 ml of ethyl acetate was added, 440 μL of 1M maleic acid solution was slowly added while stirring at 50° C., reacted at this temperature for 4 h, the solution was a turbid; slowly cooled down to 0° C. after 4 h, solid precipitation increased; the solid was obtained by centrifugal separation, the solvent is evaporated to obtain a solid product. The powder X-ray diffraction pattern of the obtained crystal is shown in FIG. 4 (20 angle has been marked), the crystal form is defined as crystal form B in this application. Its DSC and TGA patterns are shown in FIGS. 5 and 6. The crystal form B has no obvious endothermic and exothermic peaks. TGA results showed that the crystal form lost 2.941% before 100° C., and lost 1.214% from 111.97-165.04° C., and lost 6.395% from 165.04-295.43° C.

### Example 4 Preparation of Crystal Form A to Crystal Form G of Compound of Formula X

20 mg of the starting sample of free base prepared according to the method of Example 1 was dissolved in the corresponding solvent with a certain volume, and the corresponding acid was added to the 5 ml sample bottle with the molar ratio to the free base of 1.2:1. Reacted at 50° C. for 4 h, slowly cooled down to 4° C. to precipitate solids, the solid was collected by centrifugation. If the solution was still clear, the addition of anti-solvent (MTBE) was tried to induce crystallization. The obtained solid was dried at 50° C. overnight for XRPD testing. The test process and results are shown in Tables 1 and 2.

TABLE 1

| | Solvent | | | |
|---|---|---|---|---|
| Acid | EtOH(1.5 ml) | THF(2 ml) | ACE(5 ml) | EA(5 ml) |
| Hydrochloric | the solution was | the solution was | the solution was | the solution was |
| acid | clarified after | clarified after | still turbid after | still turbid after |
| | ultrasonic dissolution, | heating and | heating and | heating and |
| | no solid precipitation | ultrasonication, | ultrasonication, | ultrasonication, |
| | was observed during | and solid | solid | solid |
| | slow cooling, solids | precipitated after | precipitation | precipitation |
| | precipitate after | slow cooling | increased | increased |
| | adding anti-solvent | | during slow | during slow |
| | | | cooling | cooling |
| Sulfuric acid | the solution was | the solution was | the solution was | the solution was |
| | clarified after | clarified after | still turbid after | still turbid after |
| | ultrasonic dissolution, | heating and | heating and | heating and |
| | no solid precipitation | ultrasonication, | ultrasonication, | ultrasonication, |
| | was observed during | and solid | solid | solid |
| | slow cooling, solids | precipitated after | precipitation | precipitation |
| | precipitate after | slow cooling | increased | increased |
| | adding anti-solvent | | during slow | during slow |
| | | | cooling | cooling |
| Hydrobromic | the solution was | the solution was | the solution was | the solution was |
| acid | clarified after | clarified after | still turbid after | still turbid after |
| | ultrasonic dissolution, | heating and | heating and | heating and |
| | no solid precipitation | ultrasonication, | ultrasonication, | ultrasonication, |
| | was observed during | and solid | solid | solid |
| | slow cooling, solids | precipitated after | precipitation | precipitation |
| | precipitate after | slow cooling | increased | increased |
| | adding anti-solvent | | during slow | during slow |
| | | | cooling | cooling |
| Phosphoric acid | the solution was | the solution was | the solution was | the solution was |
| | clarified after | still turbid after | still turbid after | still turbid after |
| | ultrasonic dissolution, | heating and | heating and | heating and |
| | no solid precipitation | ultrasonication, | ultrasonication, | ultrasonication, |
| | was observed during | solid | solid | solid |
| | slow cooling, solids | precipitation | precipitation | precipitation |
| | precipitate after | increased | increased | increased |
| | adding anti-solvent | during slow | during slow | during slow |
| | | cooling | cooling | cooling |
| Methanesulfonic | the solution was | the solution was | the solution was | the solution was |
| acid | clarified after | still turbid after | clarified after | still turbid after |
| | ultrasonic dissolution, | heating and | heating and | heating and |
| | no solid precipitation | ultrasonication, | ultrasonication, | ultrasonication, |
| | was observed during | solid | and solid | solid |
| | slow cooling, solids | precipitation | precipitated | precipitation |
| | precipitate after | increased | after slow | increased |
| | adding anti-solvent | during slow | cooling | during slow |
| | | cooling | | cooling |
| Maleic acid | the solution was | the solution was | the solution was | the solution was |
| | clarified after | clarified after | clarified after | still turbid after |
| | ultrasonic dissolution, | heating and | heating and | heating and |
| | no solid precipitation | ultrasonication, | ultrasonication, | ultrasonication, |
| | was observed during | and solid | and solid | solid |
| | slow cooling, solids | precipitated after | precipitated | precipitation |
| | precipitate after | slow cooling | after slow | increased |
| | adding anti-solvent | | cooling | during slow |
| | | | | cooling |
| L-tartaric acid | the solution was | the solution was | the solution was | the solution was |
| | clarified after | clarified after | still turbid after | still turbid after |
| | ultrasonic dissolution, | heating and | heating and | heating and |
| | no solid precipitation | ultrasonication, | ultrasonication, | ultrasonication, |
| | was observed during | and solid | solid | solid |
| | slow cooling, solids | precipitated after | precipitation | precipitation |
| | precipitate after | slow cooling | increased | increased |
| | adding anti-solvent | | during slow | during slow |
| | | | cooling | cooling |
| Fumaric acid | the solution was | the solution was | the solution was | the solution was |
| | clarified after | clarified after | still turbid after | still turbid after |
| | ultrasonic dissolution, | ultrasonic dissolution, | heating and | heating and |
| | no solid precipitation | no solid precipitation | ultrasonication, | ultrasonication, |
| | was observed during | was observed during | solid | solid |
| | slow cooling, solids | slow cooling, solids | precipitation | precipitation |
| | precipitate after | precipitate after | increased | increased |
| | adding anti-solvent | adding anti-solvent | during slow | during slow |
| | | | cooling | cooling |
| Citric acid | the solution was | the solution was | the solution was | the solution was |
| | clarified after | clarified after | still turbid after | still turbid after |
| | ultrasonic dissolution, | ultrasonic dissolution, | heating and | heating and |
| | no solid precipitation | no solid precipitation | ultrasonication, | ultrasonication, |
| | was observed during | was observed during | solid | solid |
| | slow cooling, solids | slow cooling, solids | precipitation | precipitation |
| | precipitate after | precipitate after | increased | increased |
| | adding anti-solvent | adding anti-solvent | during slow | during slow |
| | | | cooling | cooling |

TABLE 2

| | Solvent | | | |
|---|---|---|---|---|
| | | Tetrahy- | | Ethyl |
| Acid | Ethanol | drofuran | Acetone | acetate |
| Hydrochloric | Amorphous | Crystal | Amorphous | Crystal |
| acid | | form A | | form A |
| Sulfuric acid | Amorphous | Amorphous | Crystal | Amorphous |
| | | | form C | |
| Hydrobromic | Amorphous | Crystal | Amorphous | Crystal |
| acid | | form D | | form D |
| Phosphoric | Amorphous | Amorphous | NA | Amorphous |
| acid | | | | |
| Methanesul- | Amorphous | Amorphous | Crystal | Crystal |
| fonic acid | | | form E | form E |
| Maleic acid | Crystal | Crystal | Crystal | Crystal |
| | form B | form B | form B | form B |
| L-tartaric | Amorphous | Amorphous | Amorphous | Crystal |
| acid | | | | form F |
| Citric acid | Amorphous | Amorphous | Crystal | Crystal |
| | | | form G | form G |
| Fumaric acid | Unsalted | Amorphous | Unsalted | Unsalted |

N/A: No solid obtained.

### Example 5 Preparation of Crystal Form I to Crystal Form III by Slow Volatilization

5 mg of the starting sample of free base prepared according to the method of Example 1 was weighed into a glass vial, the appropriate amount of the solvent in Table 3 was added respectively, after fully dissolved by ultrasonication, the solution was placed at room temperature for slow volatilization. The resulting solids were collected and sent for XRPD test after the solvent was completely volatilized, the corresponding solvents for various crystal forms are shown in Table 3 below:

TABLE 3

| Solvent | Crystal form |
|---|---|
| MeOH | Crystal form I |
| EtOH | Crystal form III |
| IPA | Crystal form I |
| THF | Crystal form I |
| 50% ACN | Crystal form II |
| ACE | Crystal form I |
| EA | Crystal form I |
| H₂O | Crystal form I |

### Example 6 Preparation of Crystal Form I by Slow Cooling

5 mg of the starting sample of free base prepared according to the method of Example 1 was weighed into a glass vial, the appropriate amount of solvent in Table 4 was added under water bath at 60° C., stirred to dissolve to obtain a nearly saturated solution, turn off the heating button and let it cool down slowly. After cooling to room temperature, the solution was placed under an ice bath and continued to cool to about 4° C. The collected suspension was centrifuged at 10000 r/min for 10 min. The supernatant was poured, and the solid was placed at room temperature for slow volatilization overnight, the obtained solid was collected and sent for XRPD test. The test results are shown in Table 4 below:

TABLE 4

| Solvent | Crystal form |
|---|---|
| ACE | Crystal form I |
| EA | Crystal form I |

### Example 7 Preparation of Crystal Form I, Crystal Form III by Suspension Shaking

Suspension shaking test was conducted at room temperature using different solvent systems. 5 mg of the starting sample of free base prepared according to the method of Example 1 was weighed into a glass vial, the appropriate amount of organic reagents in Table 5 were added, screwed the cap, and a sealing film was used to prevent the solvent from volatilizing, shaken for 24 hours and 7 days at 25° C. and 25 r/min respectively, taken out and centrifuged at 10000 r/min for 10 min, the supernatant was poured, and the solid was placed in a oven at 50° C. for drying, the resulting solid was collected and sent for XRPD test. The test results are shown in Table 5.

TABLE 5

| | Solid crystal form | Solid crystal form |
|---|---|---|
| | formed by shaking | formed by shaking |
| Solvent | for 1 day | for 7 days |
| EtOH | Crystal form III | Crystal form III |
| 50% ACN | Crystal form I | Crystal form I |
| THF | Crystal form I | Crystal form I |
| EA | Crystal form I | Crystal form I |
| ACE | Crystal form I | Crystal form I |
| H₂O | Crystal form I | Crystal form I |
| ACN | Crystal form I | NA |
| N-heptane | Crystal form I | NA |
| MTBE | Crystal form I | NA |

N/A: No solid was obtained.

### Example 8 Polymorph Screening by Mixing Shaking

Mixing shaking test was conducted using different solvent systems. 2 mg of each crystal form of crystal form I to crystal form III was weighed into a glass vial to obtain the mixed crystals, the appropriate amount of organic reagents in Table 6 were added, screwed the cap, and a sealing film was used to prevent the solvent from volatilizing, shaken for 1 days at room temperature and 25 r/min, taken out and centrifuged at 10000 r/min for 10 min at 4° C., the supernatant was poured, and the solid was placed in a oven at 50° C. for drying, the resulting solid was collected and sent for XRPD test. The test results are shown in Table 6. Observed from the results, crystal form V is a stable crystal form.

TABLE 6

| | Solid crystal form |
|---|---|
| Solvent | formed by shaking for 1 day |
| EtOH | Crystal form III |
| IPA | Crystal form III |
| ACE | Crystal form V |
| EtOAc | Crystal form V |
| MTBE | Crystal form V |
| THF | Crystal form V |
| ACN | Crystal form V |
| H₂O | Crystal form IV |
| 50% ACN | Crystal form II |

### Example 9 Solid State Stability Experiment

20 mg of crystal form A and crystal form B were weighed and stored at 60° C. and 40° C./75% RH respectively, and another group of samples were sealed at 4° C. as a control. Detected changes in crystal form and purity at 4 and 7 days. The results are shown in Table 7, where crystal form B has good stability at 60° C. and 40° C./75% RH, and the crystal form has not changed. Crystal form A is stable at 40° C./75% RH, and the impurities increase obviously at 60° C., but the crystal form has not changed.

TABLE 7

| | | | | | | | | | | | | | | Total | | |
|---|---|---|---|---|---|---|---|---|---|---|---|---|---|---|---|---|
| | | | | | | | | | | | | | | impur- | | |
| | Storage | RRT(%) | | | | | | | | | | | | ities | Content | |
| Sample | Condition | 0.86 | 0.89 | 0.92 | 0.94 | 0.97 | 1.04 | 1.07 | 1.10 | 1.13 | 1.15 | 1.16 | 1.20 | (%) | (%) | Properties |
| Crystal | 4° C. 0 d | 0.21 | 0.11 | — | 0.31 | 0.09 | 0.42 | 0.67 | 0.42 | 0.18 | 0.38 | 0.60 | — | 3.38 | 96.62 | Dark yellow |
| form | | | | | | | | | | | | | | | | powder |
| A | 40° C./75% RH 4 d | 0.21 | 0.09 | — | 0.28 | 0.09 | 0.49 | 0.75 | 0.51 | 0.19 | 0.41 | 0.60 | 0.07 | 3.66 | 96.34 | Dark yellow |
| | | | | | | | | | | | | | | | | powder |
| | 40° C./75% RH 7 d | 0.22 | 0.09 | — | 0.31 | 0.09 | 0.47 | 0.75 | 0.32 | 0.11 | 0.38 | 0.59 | — | 3.40 | 96.60 | Dark yellow |
| | | | | | | | | | | | | | | | | powder |
| | 60° C. 4 d | 0.15 | 0.14 | — | 0.67 | 0.56 | 1.25 | 10.65 | 0.29 | 0.08 | 0.44 | 0.52 | 0.05 | 14.81 | 85.19 | Dark yellow |
| | | | | | | | | | | | | | | | | powder |
| | 60° C. 7 d | 0.11 | 0.07 | 0.22 | 0.63 | 0.66 | 1.28 | 19.89 | 0.21 | 0.05 | 0.38 | 0.51 | 0.09 | 24.10 | 75.90 | Dark yellow |
| | | | | | | | | | | | | | | | | powder |
| Crystal | 4° C. 0 d | 0.19 | 0.09 | 0.05 | 0.17 | — | 0.51 | 0.43 | 0.38 | 0.21 | 0.40 | 0.64 | — | 3.06 | 96.94 | Off-white |
| form | | | | | | | | | | | | | | | | powder |
| B | 40° C./75% RH 4 d | 0.20 | 0.08 | 0.05 | 0.16 | — | 0.45 | 0.40 | 0.47 | 0.20 | 0.41 | 0.63 | 0.07 | 3.12 | 96.88 | Off-white |
| | | | | | | | | | | | | | | | | powder |
| | 40° C./75% RH 7 d | 0.25 | 0.07 | 0.05 | 0.16 | — | 0.45 | 0.41 | 0.44 | 0.20 | 0.41 | 0.63 | — | 3.05 | 96.95 | Off-white |
| | | | | | | | | | | | | | | | | powder |
| | 60° C. 4 d | 0.14 | 0.06 | — | 0.26 | — | 0.36 | 0.43 | 0.32 | 0.13 | 0.39 | 0.59 | — | 2.66 | 97.34 | Off-white |
| | | | | | | | | | | | | | | | | powder |
| | 60° C. 7 d | 0.16 | 0.05 | — | 0.29 | — | 0.35 | 0.44 | 0.29 | 0.13 | 0.40 | 0.60 | — | 2.70 | 97.30 | Off-white |
| | | | | | | | | | | | | | | | | powder |

—: Below detection limit

### Example 10 Dynamic Solubility Experiment

In order to compare whether the solubility of the free base before and after the salt formation changes, the solubility of crystal form A and crystal form Band the free base samples in 0.1M HCl, pH 4.5, pH 6.8 buffer and water were tested at room temperature. The free base samples and the two crystal forms were plotted separately in the experiment. Subsequently, about 5 mg of the free base sample prepared according to the method of Example 1 and the two crystal solid samples were weighed separately, and the solvent (If the solvent was added dropwise to 1 ml, the solution was still not clear, then stop adding) was slowly added dropwise, shaken at room temperature for 24 h, then centrifuged, the supernatant was taken, the sample was filtered with a 0.45 μm filter membrane, and the solubility was tested. The results are shown in Table 8 (the unit of concentration is mg/ml), the solubility of the two crystal forms at pH 4.5, pH 6.8 and water is significantly different from that of the free base sample, indicating that the solubility has been significantly improved after salt formation.

TABLE 8

| | Solution | | | | | | | | | | | |
|---|---|---|---|---|---|---|---|---|---|---|---|---|
| | pH 4.5 | | | pH 6.8 | | | 0.1M HCl | | | H₂O | | |
| Crystal | | | Final | | | Final | | | Final | | | Final |
| form | Solubility | Phenomenon | pH | Solubility | Phenomenon | pH | Solubility | Phenomenon | pH | Solubility | Phenomenon | pH |
| Free base | 3.59 | turbid | 5.84 | 0.84 | turbid | 6.74 | >50.05 | clear | 4.56 | 0.06 | turbid | 8.32 |
| Crystal | ≥43.8 | clear | NA | ≥74.6 | clear | NA | ≥62.8 | clear | NA | ≥81.4 | clear | NA |
| form A | | | | | | | | | | | | |
| Crystal | ≥3.07 | clear | 4.91 | ≥3.72 | clear | 6.53 | ≥30.4 | clear | NA | 3.13 | turbid | 5.06 |
| form B | | | | | | | | | | | | |

NA: The amount of solution is too small to detect

### Example 11 In Vitro Methyltransferase Assay

Recombinant PRC2 (EZH2-Y641F) was purchased from Active motif, S-adenosyl-methionin chloride (SAM) and Poly-L-lysine (PLL) were purchased from Sigma-Aldrich, H3(1-50)K27me1 peptide was purchased from Cisbio. LANCEUltra (Perkinelmer) system was used for detection. For the enzyme activity test, tested compounds are 1:3 diluted for 8-points, and added into each well, then 100 ng enzyme is added. And then the buffer [20 mM Tris pH8.5, 2 mM MgCl2, 0.01% Tween-20, 1 mM TCEP] containing 2.5 M SAM/250 nM H3 (1-50)K27me1 premixtures are added. After 3 hours enzyme reaction at RT, the premixed detection buffer (PLL, Europium antibody and Ulight) is added, and react for 1 h at RT, then read fluorescence on Tecan infinite pro. IC50s are counted by XLfit using a four-parameter model. The results are shown in Table 9:

TABLE 9
Inhibitory Activity of Compounds Against EZH2 Y641F

| | EZH2 | | EZH2 | | EZH2 |
|---|---|---|---|---|---|
| Com- | Y641F | Com- | Y641F | Com- | Y641F |
| pound | IC₅₀(μM) | pound | IC₅₀(μM) | pound | IC₅₀(μM) |
| Free base | 0.012 | D1 | >10 | D2 | 4.820 |
| D3 | >10 | D4 | >10 | | |

### Example 12 Cell Proliferation Assay

Cell lines Pfeiffer (CRL-2632), suDHL-6 (CRL-2959) and suDHL-10 (CRL-2963) were obtained from ATCC. All cell lines were cultured in RPMI-1640 medium (Gibco) plus 10% FBS (Gibco). Cells are harvested by centrifuge and densities are determined using a CounterStar cell counter. Appropriate number of cells are plated in a96-well culture plate and pre-incubate overnight. Tested compounds are 1:3 diluted for 8-points and added into each well. After 6 days' growth, cell counting kit-8 (Dojindo) is used to detect cell viability and read on Tecan infinite pro. IC50s are counted by XLfit using a four-parameter model. The results are shown in Table 10:

TABLE 10
Inhibitory Activity of Compounds Against Pfeiffer cells

| Com- | Pfeiffer | Com- | Pfeiffer | Com- | Pfeiffer |
|---|---|---|---|---|---|
| pound | IC₅₀(μM) | pound | IC₅₀(μM) | pound | IC₅₀(μM) |
| Free base | 0.007 | D1 | >10 | D2 | >3 |
| D3 | >3 | D4 | >3 | | |

It can be seen from Table 9 and Table 10 that the free base of the present invention has high inhibitory activity against EZH2 enzymes and cells. It was found that the types of substituents at positions 5 and 7 in the free base structure had a great influence on the enzyme inhibitory activity of the compounds. When the 7-position substituent is hydrogen and the 5-position is alkyl (such as free base), it has good inhibitory activity against the enzyme, but when the 7-position substituent is alkyl and the 5-position is hydrogen (such as D1-D4), the inhibitory activity against the enzyme is greatly reduced.

### Example 13 Pharmaceutical Composition

Tablet of crystal form B is prepared from the following components:

| | | |
|---|---|---|
| Crystal form B | 15 | g |
| starch | 40 | g |
| lactose | 37 | g |
| PVP | 3 | g |
| Sodium carboxymethyl starch | 3 | g |
| Sodium dodecyl sulfate | 1 | g |
| Magnesium stearate | 1 | g |

The crystal form B and starch are mixed and sieved, and then well mixed with the above other components, and tablets were compressed directly according to a conventional method.

### Example 14 Pharmaceutical Composition

Capsule of crystal form V is prepared from the following components:

| | | |
|---|---|---|
| Crystal form V | 20 | g |
| starch | 40 | g |
| lactose | 32 | g |
| PVP | 3 | g |
| Sodium carboxymethyl starch | 3 | g |
| Sodium dodecyl sulfate | 1 | g |
| Magnesium stearate | 1 | g |

The crystal form V and starch are mixed and sieved, and then well mixed with the above other components, and filled into ordinary gelatin capsules according to a conventional method.

All publications mentioned herein are incorporated by reference as if each individual document was cited as a reference, as in the present application. It should also be understood that, after reading the above teachings of the present invention, those skilled in the art can make various changes or modifications, equivalents of which falls in the scope of claims as defined in the appended claims.

1. A pharmaceutically acceptable salt of a compound of formula X, 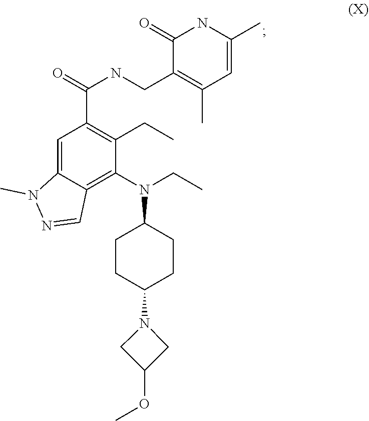
a polymorph of the compound of formula X, or a polymorph of the pharmaceutically acceptable salt of the compound of formula X,
wherein the pharmaceutically acceptable salt is selected from the group consisting of hydrochloride, sulfate, phosphate, maleate, fumarate, L-tartrate, citrate, methanesulfonate and hydrobromide.

2. The pharmaceutically acceptable salt of the compound of formula X, the polymorph of the compound of formula X, or the polymorph of the pharmaceutically acceptable salt of the compound of formula X according to claim 1, wherein the pharmaceutically acceptable salt is selected from the group consisting of hydrochloride, sulfate, maleate, L-tartrate, citrate, methanesulfonate and hydrobromide.

3. The pharmaceutically acceptable salt of the compound of formula X, the polymorph of the compound of formula X, or the polymorph of the pharmaceutically acceptable salt of the compound of formula X according to claim 1, wherein the polymorph is selected from the group consisting of
Form A crystal of the hydrochloride of the compound of formula X, i.e. crystal form A, X-ray powder diffraction pattern of which has peaks at diffraction angles 2θ(°) values of the following group A1: 4.64±0.20, 9.31±0.20, 12.11±0.20, 12.45±0.20, 13.24±0.20, 14.44±0.20, 15.28±0.20, 16.24±0.20, 16.42±0.20, 22.63±0.20, 37.87±0.20;
Form B crystal of the maleate of the compound of formula X, i.e. crystal form B, X-ray powder diffraction pattern of which has peaks at diffraction angles 2θ(°) values of the following group B1: 5.95±0.20, 13.96±0.20, 16.09±0.20, 16.42±0.20, 17.77±0.20, 19.03±0.20, 20.29±0.20, 20.65±0.20, 21.73±0.20;
Form C crystal of the sulfate of the compound of formula X, i.e. crystal form C, X-ray powder diffraction pattern of which has peaks at diffraction angles 2θ(°) values of the following group C1: 7.00±0.20, 13.18±0.20, 14.14±0.20, 14.44±0.20, 14.62±0.20, 17.65±0.20, 17.81±0.20, 18.11±0.20, 20.44±0.20, 21.85±0.20, 23.89±0.20;
Form D crystal of the hydrobromide of the compound of formula X, i.e. crystal form D, X-ray powder diffraction pattern of which has peaks at diffraction angles 2θ(°) values of the following group D1: 14.65±0.20, 16.47±0.20, 17.62±0.20, 17.92±0.20, 21.91±0.20, 22.99±0.20, 23.12±0.20, 24.88±0.20;
Form E crystal of the methanesulfonate of the compound of formula X, i.e. crystal form E, X-ray powder diffraction pattern of which has peaks at diffraction angles 2θ(°) values of the following group E1: 9.48±0.20, 10.30±0.20, 12.03±0.20, 12.79±0.20, 13.90±0.20, 16.09±0.20, 16.39±0.20, 17.76±0.20, 18.97±0.20, 19.11±0.20, 20.08±0.20, 20.39±0.20, 20.59±0.20, 21.73±0.20, 21.91±0.20, 22.14±0.20, 22.99±0.20, 23.14±0.20, 23.57±0.20, 25.67±0.20;
Form F crystal of the L-tartrate of the compound of formula X, i.e. crystal form F, X-ray powder diffraction pattern of which has peaks at diffraction angles 2θ(°) values of the following group F1: 10.92±0.20, 11.11±0.20, 11.26±0.20, 15.31±0.20, 16.96±0.20, 17.11±0.20, 18.16±0.20, 18.46±0.20, 20.45±0.20, 23.55±0.20, 25.30±0.20;
Form G crystal of the citrate of the compound of formula X, i.e. crystal form G, X-ray powder diffraction pattern of which has peaks at diffraction angles 2θ(°) values of the following group G1: 11.11±0.20, 15.22±0.20, 16.99±0.20, 18.16±0.20, 20.47±0.20, 23.26±0.20, 23.44±0.20;
crystal form I of the compound of formula X, X-ray powder diffraction pattern of which has peaks at diffraction angles 2θ(°) values of the group I-1: 7.09±0.20, 9.58±0.20, 11.17±0.20, 13.40±0.20, 14.02±0.20, 14.65±0.20, 16.51±0.20, 17.59±0.20;
crystal form II of the compound of formula X, X-ray powder diffraction pattern of which has peaks at diffraction angles 2θ(°) values of the following group II-1: 5.32±0.20, 7.11±0.20, 9.16±0.20, 9.56±0.20, 11.15±0.20, 11.62±0.20, 13.45±0.20, 14.02±0.20, 14.65±0.20, 16.54±0.20, 17.62±0.20, 21.91±0.20, 37.96±0.20, 38.38±0.20, 44.20±0.20;
crystal form III of the compound of formula X, X-ray powder diffraction pattern of which has peaks at diffraction angles 2θ(°) values of the following group III-1: 6.97±0.20, 9.01±0.20, 9.21±0.20, 12.46±0.20, 14.86±0.20, 15.28±0.20, 15.46±0.20, 20.92±0.20, 22.90±0.20;
crystal form IV of the compound of formula X, X-ray powder diffraction pattern of which has peaks at diffraction angles 2θ(°) values of the following group IV-1: 5.26±0.20, 6.94±0.20, 8.98±0.20, 9.52±0.20, 11.62±0.20, 12.40±0.20, 13.42±0.20, 14.62±0.20, 16.51±0.20, 17.62±0.20, 18.01±0.20, 19.18±0.20, 21.85±0.20, 27.31±0.20; and
crystal form V of the compound of formula X, X-ray powder diffraction pattern of which has peaks at diffraction angles 2θ(°) values of the following group V-1: 7.00±0.20, 8.98±0.20, 9.55±0.20, 11.11±0.20, 12.46±0.20, 13.42±0.20, 14.68±0.20, 15.25±0.20, 15.45±0.20, 17.59±0.20, 19.30±0.20, 20.86±0.20, 21.88±0.20, 22.99±0.20, 27.49±0.20, 37.90±0.20.

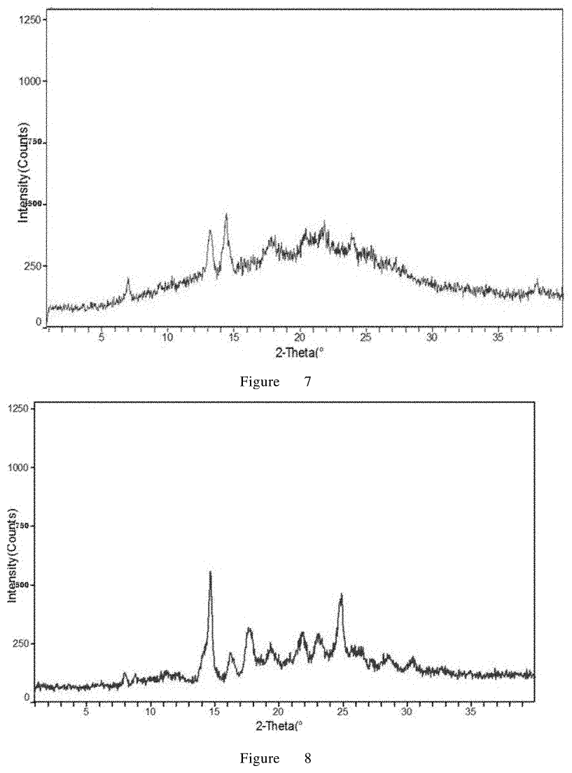

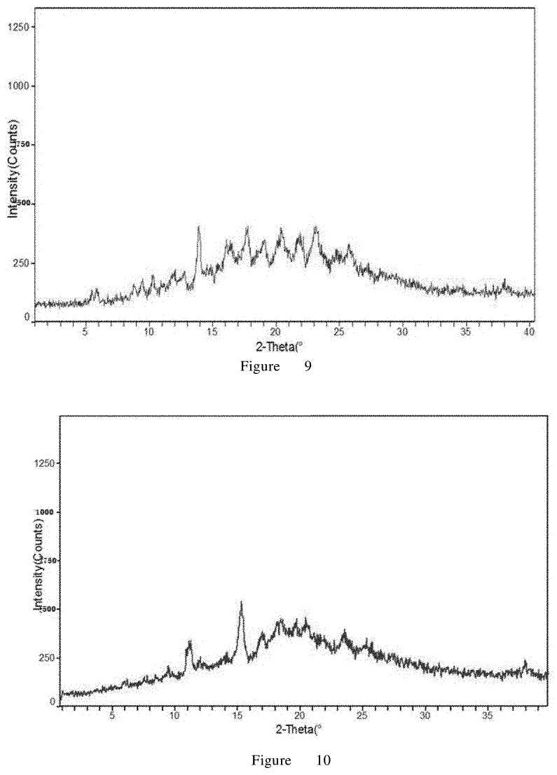

4. The pharmaceutically acceptable salt of the compound of formula X, the polymorph of the compound of formula X, or the polymorph of the pharmaceutically acceptable salt of the compound of formula X according to claim 3, wherein
the X-ray powder diffraction pattern of the crystal form A is substantially as shown in FIG. 1;
the X-ray powder diffraction pattern of the crystal form B is substantially as shown in FIG. 4;
the X-ray powder diffraction pattern of the crystal form C is substantially as shown in FIG. 7;
the X-ray powder diffraction pattern of the crystal form D is substantially as shown in FIG. 8;
the X-ray powder diffraction pattern of the crystal form E is substantially as shown in FIG. 9;
the X-ray powder diffraction pattern of the crystal form F is substantially as shown in FIG. 10;
the X-ray powder diffraction pattern of the crystal form G is substantially as shown in FIG. 11.

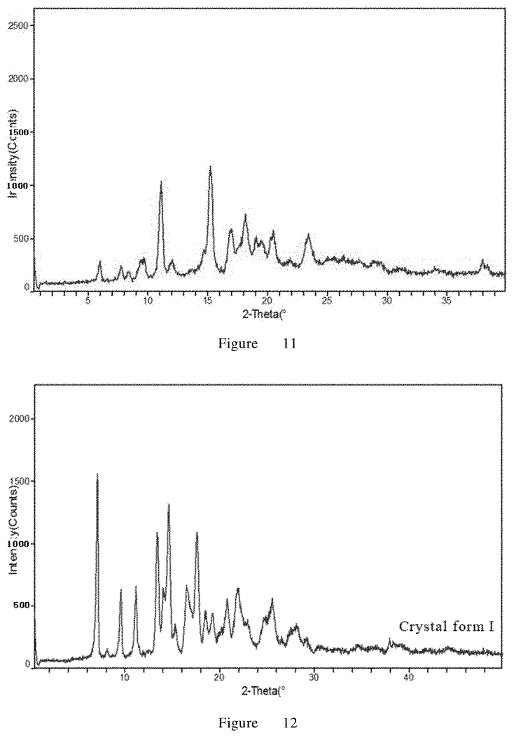

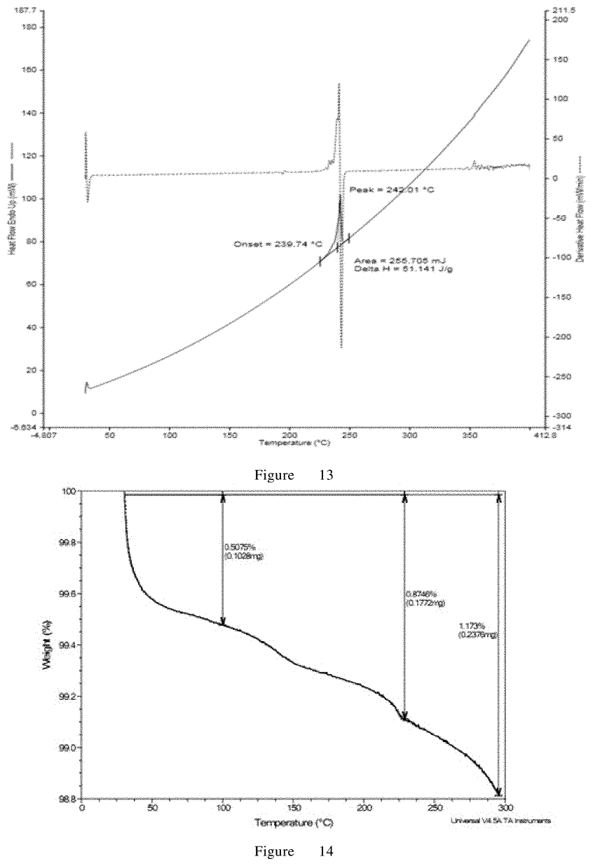

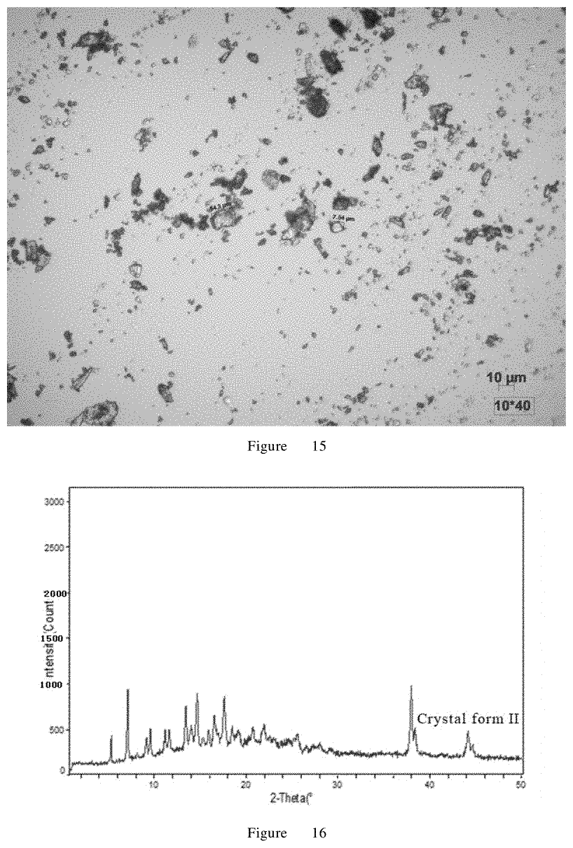

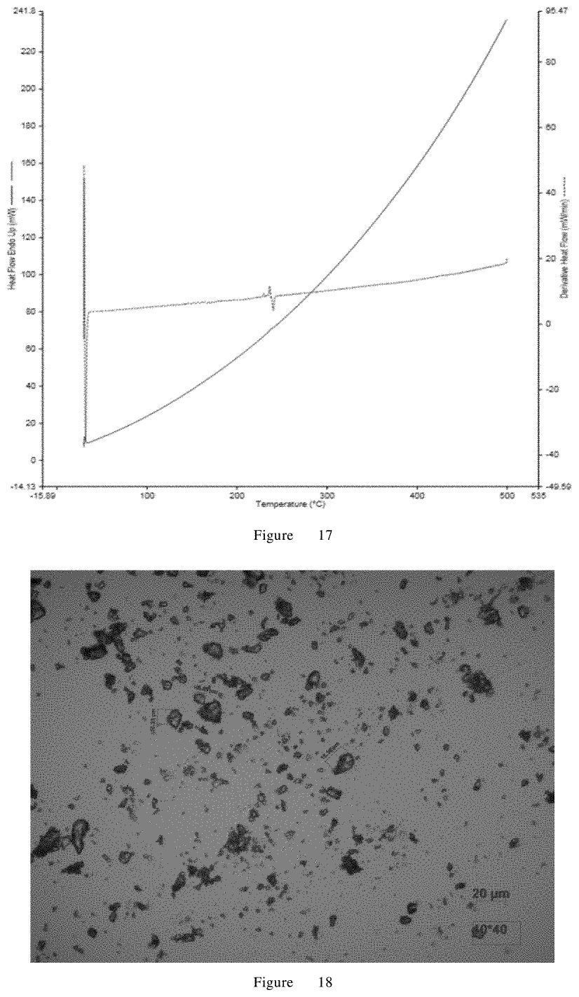

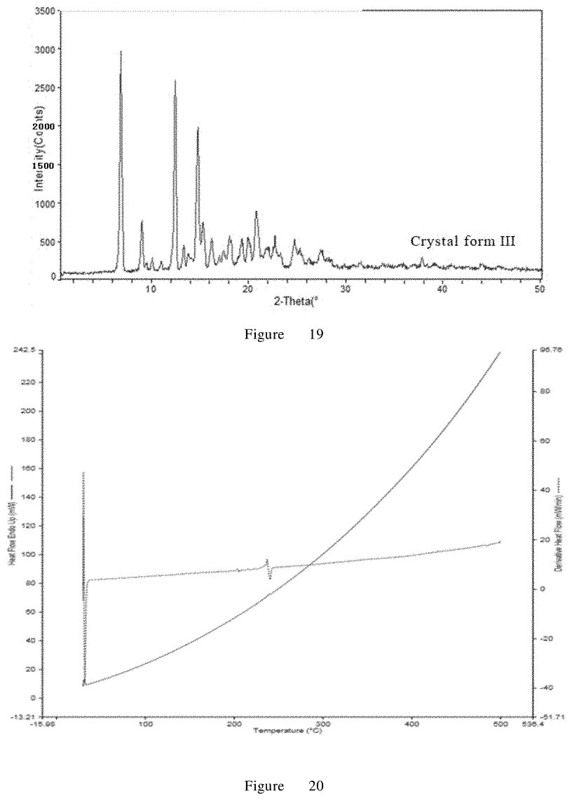

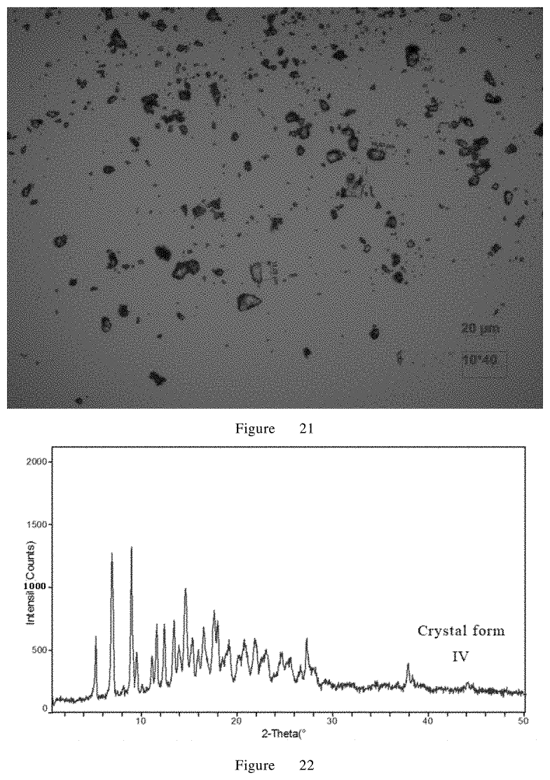

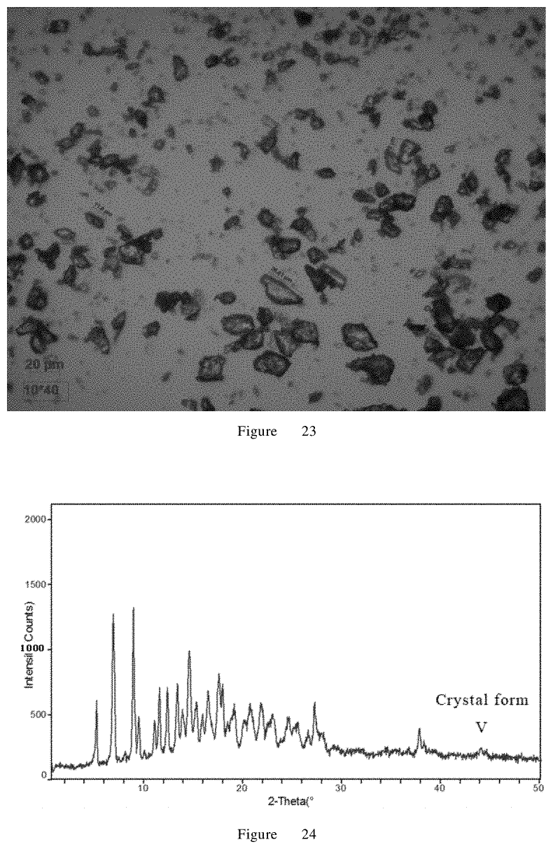

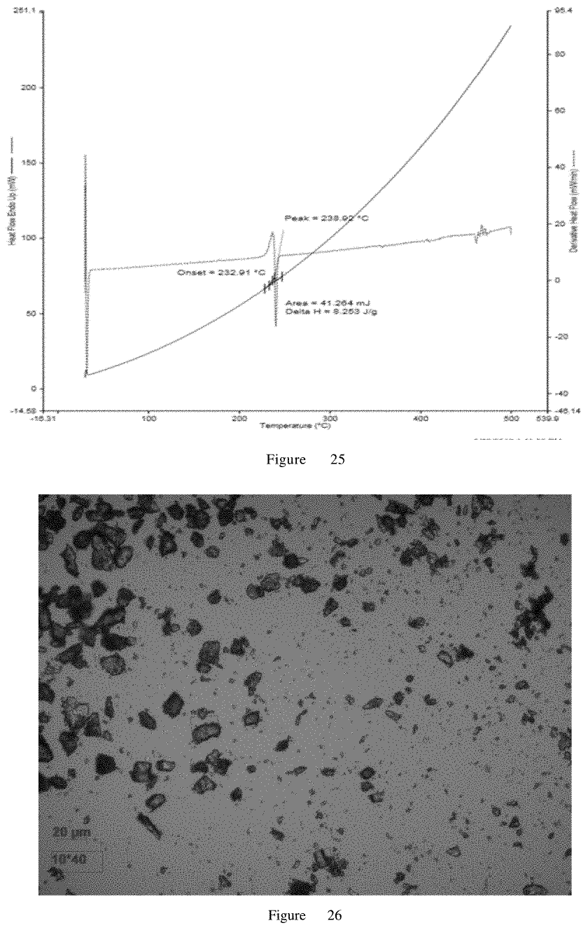

5. The pharmaceutically acceptable salt of the compound of formula X, the polymorph of the compound of formula X, or the polymorph of the pharmaceutically acceptable salt of the compound of formula X according to claim 3, wherein
the X-ray powder diffraction pattern of the crystal form I is substantially as shown in FIG. 12;
the X-ray powder diffraction pattern of the crystal form II is substantially as shown in FIG. 16;
the X-ray powder diffraction pattern of the crystal form III is substantially as shown in FIG. 19;
the X-ray powder diffraction pattern of the crystal form IV is substantially as shown in FIG. 22;
the X-ray powder diffraction pattern of the crystal form V is substantially as shown in FIG. 24.

6. A process for preparing a pharmaceutically acceptable salt of a compound of formula X, a polymorph of the compound of formula X, or a polymorph of the pharmaceutically acceptable salt of the compound of formula X, wherein the process comprises steps of:
(1) reacting a compound 5a with a compound 1a in a solvent to form the compound of formula X; 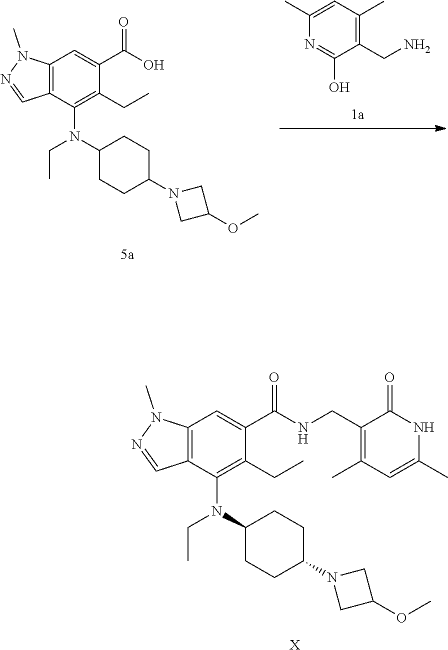
and
(2) optionally, conducting a salt-forming reaction with the compound of formula X and an acid to form a pharmaceutically acceptable salt;
(3) optionally, crystallizing the compound of formula X formed in the step (1) or the pharmaceutically acceptable salt formed in the step (2) to obtain a polymorph.

7. A pharmaceutical composition, wherein the pharmaceutical composition comprises:
(a) the pharmaceutically acceptable salt of the compound of formula X, the polymorph of the compound of formula X, or the polymorph of the pharmaceutically acceptable salt of the compound of formula X according to claim 1; and
(b) a pharmaceutically acceptable carrier.

8. Use of the pharmaceutically acceptable salt of the compound of formula X, the polymorph of the compound of formula X, or the polymorph of the pharmaceutically acceptable salt of the compound of formula X according to claim 1, in the preparation of a drug for an EZH2-mediated disease or condition.

9. The use according to claim 8, wherein the EZH2-mediated disease or condition is selected from the group consisting of cancer, pulmonary arterial hypertension, myelofibrosis, human immunodeficiency virus (HIV) disease, graft versus host disease (GVHD), Weaver syndrome, psoriasis vulgaris and liver fibrosis.

10. Use of the pharmaceutical composition according to claim 7 in the preparation of a drug for an EZH2-mediated disease or condition.

11. The use according to claim 10, wherein the EZH2-mediated disease or condition is selected from the group consisting of cancer, pulmonary arterial hypertension, myelofibrosis, human immunodeficiency virus (HIV) disease, graft versus host disease (GVHD), Weaver syndrome, psoriasis vulgaris and liver fibrosis.
